(19) United States
(12) Patent Application Publication
(10) Pub. No.: US 2015/0112188 A1
(43) Pub. Date: Apr. 23, 2015

(54) SYSTEMS AND METHODS FOR MONITORING ENDOLUMINAL VALVE FORMATION

(71) Applicant: VOLCANO CORPORATION, San Diego, CA (US)

(72) Inventors: Jeremy Stigall, Carlsbad, CA (US); David Goodman, Carlsbad, CA (US)

(21) Appl. No.: 14/491,383

(22) Filed: Sep. 19, 2014

Related U.S. Application Data
(60) Provisional application No. 61/880,532, filed on Sep. 20, 2013.

(51) Int. Cl. A61B 17/00; A61B 17/22; A61B 8/00; A61B 5/00; A61B 8/08; A61B 8/12

(52) U.S. Cl. CPC A61B 17/00234; A61B 8/0841; A61B 8/12; A61B 8/4494; A61B 5/0095; A61B 17/22032; A61B 8/445; A61B 5/6853; A61B 2017/00261; A61B 2017/00783; A61B 2017/22097
USPC 600/424

(57) ABSTRACT

The invention generally relates to systems for imaging and monitoring endoluminal valve formation. In certain aspects, a system of the invention includes: an elongate body defining a lumen and an exit port located on a side of the elongate body, wherein the side of the elongate body is configured to engage with a vessel wall; a tissue dissection probe disposed within the lumen, and configured to extend out of the exit port and into the vessel wall in order to form an intramural space in the vessel wall; and an imaging element located on the tissue dissection probe.

### RELATED APPLICATION

This application claims the benefit of and priority to U.S. Provisional No. 61/880,532, filed Sep. 20, 2013, which is incorporated by reference herein.

### TECHNICAL FIELD

This application relates generally to systems and methods for monitoring endoluminal valve formation.

### BACKGROUND

The venous system returns blood to the heart from the rest of the body. In healthy individuals, natural valves within veins permit blood flow in a substantially unidirectional manner along the length of the vessels. These one-way valves keep blood flowing toward the heart, against the force of gravity while preventing backflow.

Venous insufficiency is a condition in which the flow of blood through the veins is impaired, typically due to valve malfunction. When a valve malfunctions, blood may backflow into an extremity, such as a leg, causing blood pooling and distention. The pooling of blood caused by venous insufficiency leads to increased pressure or hypertension within the veins. The symptoms associated therewith include pain, swelling, and ulcers in the affected extremity. Elevation of the feet and compression stockings can relieve some symptoms, but do not treat the underlying disease. Untreated, the disease can impact the ability of individuals to maintain their normal lifestyle.

In order to treat venous insufficiency, a number of surgical procedures have been employed to improve or replace the native valve, including the implantation of prosthetic valves. A prosthetic valve is designed to mimic a natural valve in order to regain unidirectional blood flow within a vein. However, prosthetic valves are prone to high failure rates and biocompatibility issues. Due to these issues, implantation of a prosthetic valve is typically a last resort. As an alternative to prosthetic valves, other surgical procedures are being explored to create an autologous valve formed from an intimal tissue flap of a vessel wall. In order to create an autologous valve, an intimal tissue flap is created within a vessel wall, and then secured within the vessel such that the tissue flap mimics the function of a native valve. While lacking the risk of biocompatibility associated with prosthetic valves, autologous valve formation is a complex surgery that includes the inherent risk of tearing or puncturing the vessel wall. As such, there is a need for improving the systems and methods used to form valves in veins.

### SUMMARY

The invention recognizes that current autologous valve formation procedures are limited because prior art valve formation devices do not allow visualization of the procedure within the lumen. Without visualization, the risk of puncturing the vessel or tearing the vessel wall is increased. Systems and methods of the invention reduce risk associated with autologous valve formation by providing systems that incorporate imaging with a catheter and/or a dissection probe used to form the intimal tissue flap. Such systems allow an operator to visualize the intimal flap and surrounding vessel surfaces while the intimal flap is being formed. In addition, systems of the invention may also include pressure and flow sensors that alert the operator of abnormal pressure/flow changes within the vessel during the autologous valve formation procedure. The abnormal pressure/flow changes may signify undesirable vessel puncture.

Systems of the invention include a support catheter and a tissue dissection probe (also referred to as puncture members) that extends out of the support catheter and into a vessel wall. Once disposed within the vessel wall, the tissue dissection probe is able to separate an inner tissue layer from the vessel wall to form a tissue flap. The tissue dissection probe may eject hydro-dissection fluid into the intramural space of the vessel wall to separate the tissue layers and thereby form the tissue flap. In particular embodiments, the tissue dissection probe also forms a pouch within the intramural space of the vessel wall using an expansion member. The expansion member forms a tissue flap of a certain shape ideal for valve creation. The support catheter, the tissue dissection probe, or both may include an imaging sensor, a functional measurement sensor, or a combination thereof.

The catheter systems of the invention, equipped with imaging elements, functional measurement sensors or both, can advantageously provide for 1) real-time imaging of intraluminal surfaces to detect a location of interest, 2) forming an intimal tissue flap within a vessel wall at the location of interest, 3) forming an endoluminal valve with the intimal tissue flap 3) real-time imaging of the location of interest (e.g. various vessel surfaces) before, during, and after the endoluminal procedure, and 4) real-time measurement of function parameters (such as pressure, flow, and temperature) before, during, and after the endoluminal valve formation procedure.

As discussed, the systems of the invention may include one or more imaging elements. Imaging elements of the invention may be a forward-looking imaging element, side-looking element, or combination of the two. Suitable imaging elements include, for example, ultrasound transducers and photoacoustic transducers. In addition, systems of the invention may include one or more functional measurement sensors. Functional measurement sensors include pressure sensors, flow sensors, temperature sensors, or combinations thereof. In one embodiment, the imaging element is placed on a distal portion of the support catheter. In another embodiment, the imaging element is placed on a distal portion of a dissection probe that enters the intramural space of a vessel wall. The imaging element of the dissection probe may be placed on or beneath an expansion member used to create a pocket within the vessel wall. Likewise, a functional measurement sensor may be placed on a distal portion of the support catheter or on a distal portion of a tissue dissection probe.

According to certain aspects, systems of the invention include at least two expandable members coupled to a support catheter so that the expansion members can stabilize the support catheter within the vessel during the endoluminal valve formation procedure. The expansion members provide bipod support while pressing the side of the support catheter where the tissue dissection device is deployed against the vessel wall. In this manner, the tissue dissection device is deployable into the vessel wall from the support catheter in a controlled manner without risk of unwanted movement.

### BRIEF DESCRIPTION OF THE DRAWINGS

FIG. 1A depicts a support catheter of a valve-forming catheter system pressed against a vessel wall according to certain embodiments.

FIG. 1B depicts a top view of FIG. 1A at line A-A.

FIG. 1C depicts the support catheter of FIG. 1A pressed against the vessel wall at an angle.

FIG. 1D depicts an embodiment of the support catheter with imaging and functional measurement elements.

FIG. 1E depicts an embodiment of the support catheter with imaging and functional measurement elements.

FIG. 1F illustrates an exit port offset from a rigid distal portion of a valve-forming catheter.

FIGS. 1G-1J depict a puncture element deployed from the support catheter and desirable dimensions between the deployed puncture element and a distal portion of the support catheter.

FIGS. 2A-2D depict various dissection probes and puncture elements suitable for use with valve-forming catheter systems of the invention.

FIGS. 3A-3C depict alternative expansion members coupled to a support catheter according to certain embodiments.

FIGS. 4A-4B depict deployment of a puncture element from the support catheter of a valve-forming catheter system.

FIGS. 5A-5B depict pouch formation within a vessel wall according to certain embodiments.

FIGS. 6A-6B depict pouch formation within in a vessel wall using an puncture element with an expansion member.

FIGS. 7A-7B depicts an alternative puncture element with a fluid dissection element with and without an expansion member.

FIGS. 8A-8B illustrate top views of autologous monocuspid valves in the open configuration (blood flowing up), and attached to the vessel wall with alternate embodiments of securing.

FIGS. 8C-8D illustrate top views of autologous bicuspid valves in the open configuration (blood flowing up), and attached to the vessel wall with alternate embodiments of securing.

FIGS. 9A-9E depict a support catheter of a valve-forming catheter system according to certain embodiments.

FIGS. 10A-10C depict a guide member of a valve-forming catheter system according to certain embodiments.

FIGS. 11A-11B depict a tissue dissection probe of a valve-forming catheter system according to certain embodiments.

FIGS. 12A-12G depict a tissue dissection probe and securement mechanism of a valve-forming catheter system according to certain embodiments.

FIGS. 13-18B depict a method of using the devices depicted in FIGS. 9A-12G within the context of a percutaneous valve creation procedure. FIG. 13 depicts the support catheter positioned at a valve creation site within a body lumen. FIG. 14A depicts the support catheter of with the wall-tensioning mechanism actuated. FIG. 14B depicts the support catheter with the angling mechanism actuated. FIGS. 15A-B depict the sub-intimal access probe being deployed from the support catheter. FIG. 16 depicts the tissue layer separation mechanism being used at the valve creation site. FIGS. 17A-17J depict use of the sub-intimal pocket probe to deploy the valve attachment mechanism. FIGS. 18A-18B depict the intimal separation mechanism being utilized.

FIG. 19 depicts a system for use with catheters and systems of the invention.

FIGS. 20A, 20B and 20C are schematic views of a valve prosthesis according to certain embodiments. FIG. 20A depicts a valve prosthesis having a support frame supporting two valve leaflets formed from a continuous membrane in the form of a cone structure. The cone structure tapers towards the downstream end of the valve prosthesis and terminates at two co-apting edges. FIG. 20B depicts a valve prosthesis having a support frame supporting two valve leaflets formed from a continuous membrane in the form of a cone structure. The cone structure is supported by two support elements and tapers towards the downstream end of the valve prosthesis and terminates at two co-apting edges. FIG. 20C depicts another valve prosthesis having a support frame supporting two valve leaflets formed from a continuous membrane in the form of a cone structure.

FIG. 21 is a schematic view of another illustrative embodiment of a valve prosthesis of the present invention.

FIGS. 22A and 22B are schematic plan views of a valve prosthesis having three valve leaflets. FIG. 22A depicts a valve prosthesis having leaflets positioned by retrograde flow. FIG. 22B depicts a valve prosthesis having leaflets positioned by antegrade flow.

FIG. 23 depicts a schematic overview of valve-forming catheter system of the invention that includes a rotatable imaging element.

### DETAILED DESCRIPTION

The present invention generally relates to catheter systems for forming endoluminal valves with imaging capabilities, functional measurement capabilities, or both. The catheter systems of the invention for forming endoluminal valves are also referred to herein as tissue dissection assemblies. The catheter systems of the invention may provide for 1) real-time imaging of intraluminal surfaces to detect a location of interest, 2) forming a tissue flap within a vessel wall at the location of interest, 3) forming an endoluminal valve with the tissue flap 3) real-time imaging of the location of interest (e.g. various vessel surfaces) before, during, and after the endoluminal procedure, and 4) real-time measurement of function parameters (such as pressure, flow, and temperature) before, during, and after the endoluminal procedure.

Typically, catheter systems of the invention include a stabilizing/support catheter and one or more puncture members (as referred to as tissue dissection probes) configured to extend out of the stabilizing catheter and into a vessel wall. As used in this specification, the term support catheter or similar terms refer to any device that provides a conduit, channel, or lumen for housing and/or delivering a component or a substance. The support catheter serves as a platform to support other device components, such as one or more puncture members, which can be inserted percutaneously into bodily lumen(s). For example, once the support catheter is positioned at a location for flap formation, the puncture member is extended into a vessel wall. When disposed within the vessel wall, the puncture member is configured to separate a tissue flap from a tissue layer of the vessel wall without puncturing the vessel wall. The support catheter, the one or more puncture members, or combinations thereof may include an imaging sensor, functional measurement sensor or combination thereof. For example, in one embodiment, the support catheter may include an imaging element and the puncture member may include a functional measurement sensor. In another example, both the support catheter and the puncture member may include an imaging element and/or functional measurement sensor. In yet another example, only the support catheter or the puncture member includes the functional measurement sensor and/or the imaging element.

Imaging elements and functional measurement sensor are described in more detail hereinafter. Briefly, the imaging element may include, for example, an ultrasound transducer or photo-acoustic transducer. The functional measurement sensor may include a flow sensor, pressure sensor, and temperature sensor.

In certain embodiments, an imaging guidewire can be introduced into a lumen of the body to obtain real-time images of the vessel prior to introduction of the support catheter over the guidewire. The body lumens generally are lumens of the vasculature. The real-time images obtained may be used to locate a region or location of interest within a body lumen. Regions of interest are typical regions that are an ideal location for forming a valve within a vessel. For example, the location may be the ideal location to form a valve within the vessel for treating venous reflux. The devices and methods, however, are also suitable for forming tissue flaps and valves in other body lumens, such as the respiratory passages, the pancreatic system, the lymphatic system, and the like.

Systems and methods of the invention are designed to enter a body lumen and form an intramural space within a wall of the body lumen. Typically, the intramural space forms a tissue flap from the extending from the vessel wall. The tissue flap can then be secured to a vessel wall to form a valve. In accordance with embodiments for creating an intra-mural potential space, and access to that space, systems of the invention include a support catheter and one or more puncture elements. The puncture element may form an intra-mural space by, for example, injecting a hydrodissection fluid into the vessel wall, expanding an expansion member located on or formed as part of the puncture element, or by a combination of said injection/expansion. The various embodiments of the support catheter and puncture elements for creating an intramural space as well as methods for creating an intra-mural space are described hereinafter. In addition, concepts of the invention may be applied to prior art systems for forming endoluminal valves, such as those described in U.S. Publication Nos. 2011/0264125 and 2012/0289987, the entireties of which is incorporated by reference herein.

The following describes generally methods for creating an intramural space in a vessel wall using support catheters and puncture elements of the invention. In accordance with some embodiments, a method includes stabilizing a support catheter against vessel wall at a desired location to form an intramural space/tissue flap in the vessel wall. Once secured, a probe (e.g. puncture element) is advanced into the vessel wall a minimal amount. The probe then expels a pressurized hydrodissection agent (saline or saline with a contrast agent, or a hydrogel, or water for injection) from its distal tip to separate the intimal tissue layer from the medial tissue layer, or the medial layer from the adventitial layer, or a fibrosis layer from the intimal layer, or a sub-medial layer from another sub-medial layer, or a sub-adventitial layer from another sub-adventitial layer. This propagates distally from the distal end of the probe. In this way, a tissue pocket is formed without the need to further advance the probe into the wall, as long as sufficient flow is provided, and the pocket created is free from a significant leak at the top of the pocket (at probe entry), or from a hole leading into the lumen or extravascular space. In this way a fluid sealed pocket is formed with only one opening at the entry point. In some embodiments, a typical hydrodissection flow is between 0.25 cc/second and 3 cc/second. In other embodiments, a typical hydrodissection flow is between 0.5 cc/second and 2 cc/second. In other embodiments, a typical hydrodissection flow is between 0.75 cc/second and 1.25 cc/second. In addition or as an alternative to injecting hydrodissection fluid, once the probe is disposed within the vessel wall, an expandable member on the probe is expanded to form the intraluminal space.

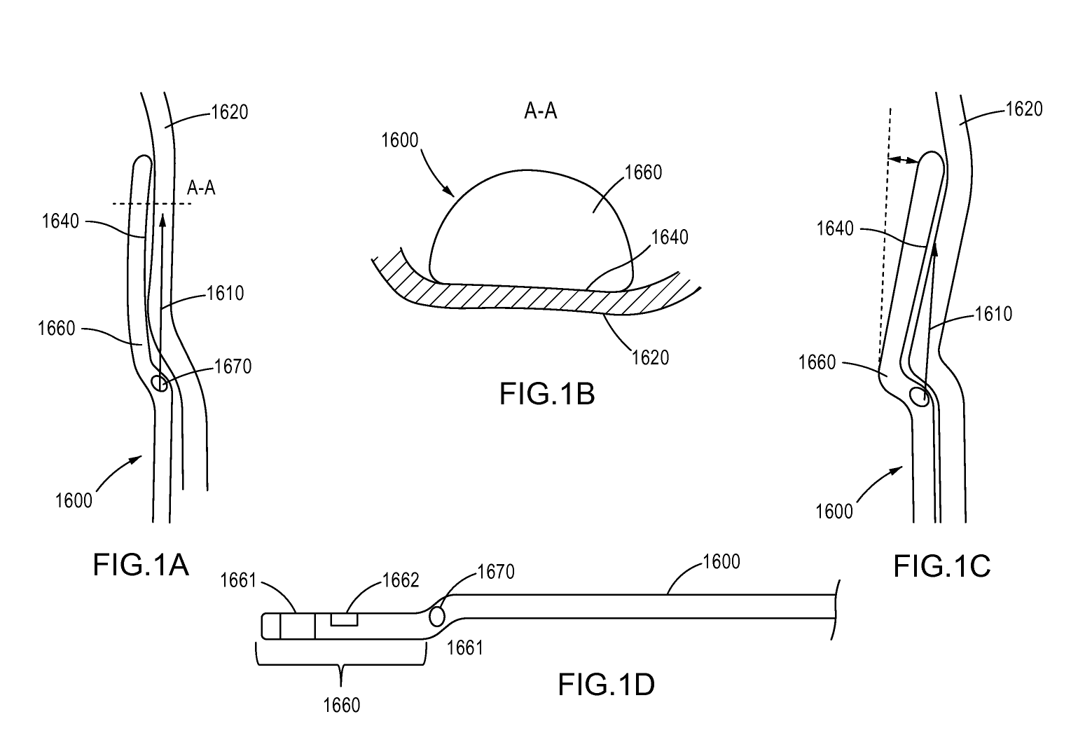

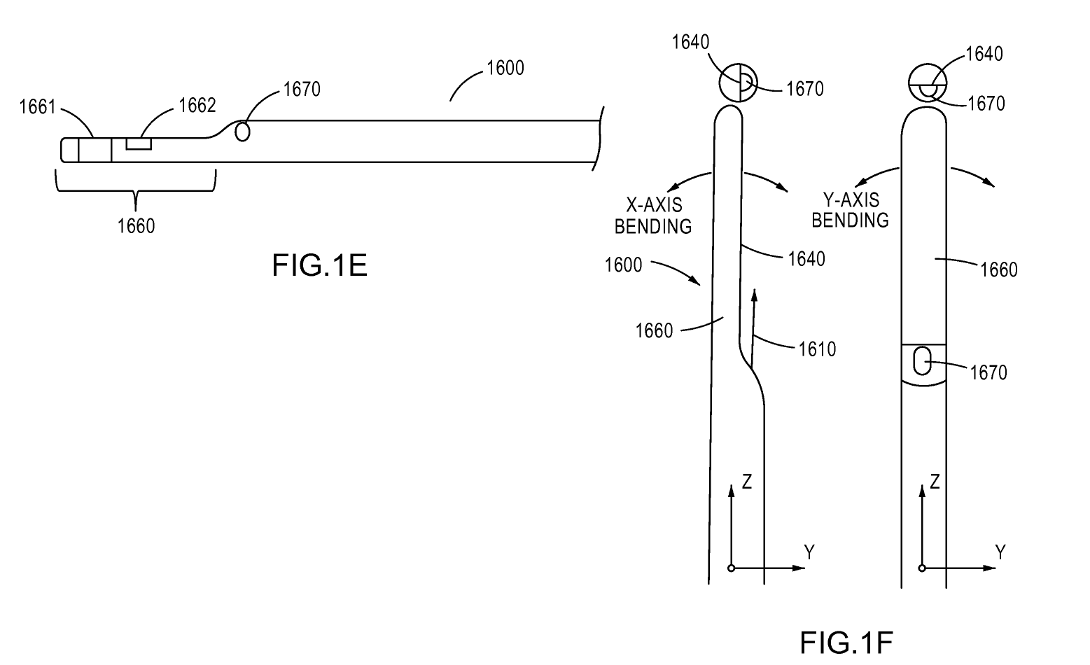

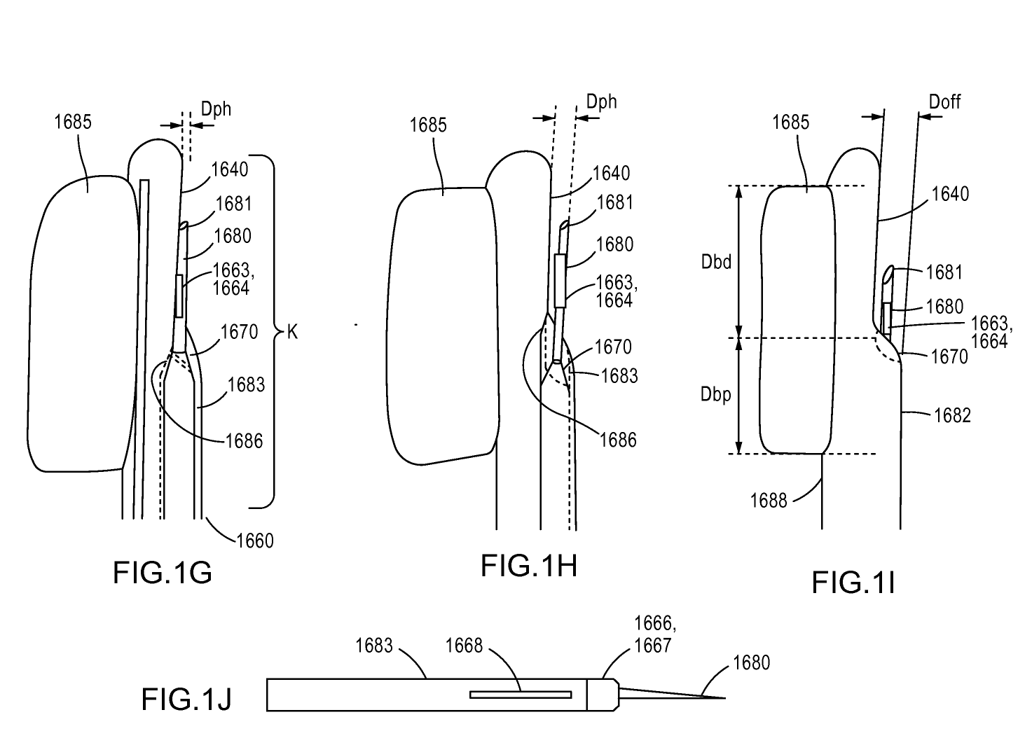

FIG. 1A depicts a support catheter for formation of an intraluminal space according to certain embodiments. As depicted in FIG. 1A, the support catheter 1600 includes a distal portion 1660. In certain embodiments, the distal portion 1660 has a flat support surface 1640, which allows it to rest firmly against the vessel wall. Proximal to the flat surface 1640 is an exit port 1670. A puncture member (described hereinafter) may be extended out of the exit port 1670 and into a vessel wall 1620 next to the distal portion 1660, following the path of the arrow. Once inserted into the vessel wall 1620, the puncture element can be used to form the intramural space within the vessel wall. As shown and preferably, distal portion 1660 is offset from the portion of the catheter where the exit port 1670 is located. The exit port 1670 is located along a side of the support catheter 1600. Preferably, the exit port 1670 located on a side of the support catheter 1600 is orientated such at the puncture member, extended out of the exit port 1670, is parallel to the distal portion 1660 (See FIG. 1F). The support catheter may have an S-shape, as shown, to achieve that orientation (See FIGS. 1C and 1D). Alternatively, the support catheter may taper at the distal portion to achieve that orientation (see FIGS. 1E and 1F). In the tapered-configuration, a cross-section of the distal portion of the support catheter is smaller than a cross-section of a portion of the support catheter proximal to the distal portion.

In accordance with some embodiments, the support catheter 1600 is described to aid in the direction of advancement 1610 of a tissue dissection probe within the vessel wall 1620 by controlling the angle of the vessel wall 1620. In one embodiment, a sufficiently stiff, flat surface 1640 along the distal portion 1660 of the support catheter 1600 exists to ensure the vessel wall 1620 does not bend inward toward the lumen, and thus preventing the advancement direction of the tissue dissection probe 1610 from pointing outward through the adventitia (FIG. 1A). This embodiment of the support catheter 1600 is shown in cross-section (at the distal portion 1660) in FIG. 1B, depicting the flatness of the flat surface 1640. The depiction shows the vessel wall 1620, which rests against the flat surface 1640, as it is made to conform to a flat orientation. The transitory portion of the tubular assembly between the distal portion and the proximal portion can be s-shaped so that the flat surface 1640 of the distal portion 1660 is offset from the port at the distal end of the proximal portion of the support catheter 1660. The degree of offset can control the penetration depth of the tissue dissection probe. For example, the offset can be between about 0.1 mm to 5 mm. In other embodiments, the offset can be between about 0.5 mm to 3 mm. In other embodiments the offset can be between about 0.75 mm and 1.5 mm. The degree of offset may be the same or similar in the tapered configuration of the support catheter 1600. A similar embodiment includes a flat surface 1640 along the distal portion of the supporting tubular assembly 1660, which is angled outward, away from the center of the lumen by between about 0° and 15°, or about 1° and 10°, or about 2° and 6°. This structure ensures that the path of the tissue dissection probe is close to the axis of the vessel wall, but with a slight bias toward the intraluminal side (FIG. 1C).

FIGS. 1D and 1E depicts preferred embodiments of the support catheter 1600 (shown in the S-configuration and in the tapered configuration, respectively). As depicted in FIGS. 1D and 1E, the support catheter includes an imaging element and a functional measurement sensor. While the support catheter 1600 is shown with both the imaging element and functional measurement sensor, it is envisioned that the support catheter may include either one by itself. The imaging element allows one to image the luminal surface of the vessel in order to determine a certain location for the intramural space in the vessel wall for valve formation. The functional measurement sensor allows one to monitor vital signs within the vessel such as blood pressure and blood flow that also allow one to determine where valves are not properly regulating blood pressure and flow, and where a valve is needed to regulate pressure and blood flow.

In certain embodiments, the stiff, flat surface 1640 of the distal portion 1660 of the support catheter 1600 is sufficiently stiff to resist bending about the x and y axis (as depicted). This way, if the vessel in which the device is implanted takes a tortuous path, the distal portion 1660 resists bending along with the vessel, which allows advancement of a puncture element or tissue dissection probe to be ensured to maintain sufficiently parallel trajectory 1610 (along the z axis) and to maintain position within the center of the flat surface 1640 (not meandering off the side of the flat surface entirely along the positive or negative x axis). This can be done by using inherently stiff materials for the entire support catheter 1600 or exclusively in the distal portion 1660 of the support catheter 1660. In some embodiments, the distal portion 1660 that must have sufficient stiffness can be defined by the portion spanning at least 4 cm proximal to the exit port 1670 of the support catheter, and spanning at least 4 cm distal the exit port 1670. In some embodiments, the distal portion 1660 that must have sufficient stiffness can be defined by the portion spanning at least 2 cm proximal to the exit port 1670 of the support catheter, and spanning at least 2 cm distal the exit port 1670. In some embodiments, the distal portion 1660 that must have sufficient stiffness can be defined by the portion spanning at least 1.25 cm proximal to the exit port 1670 of the support mechanism, and spanning at least 1.25 cm distal the exit port 1670. In some embodiments sufficiently stiff is defined as less than 4 mm of deformation if a 0.5 lb force is applied along a 6 cm lever arm. In some embodiments sufficiently stiff is defined as resistance to 2 mm of deformation if a 0.5 lb force is applied along a 6 cm lever arm. In some embodiments sufficiently stiff is defined as resistance to 1 mm of deformation if a 0.5 lb force is applied along a 6 cm lever arm. In some embodiments sufficiently stiff is defined as resistance to 0.25 mm of deformation if a 0.5 lb force is applied along a 6 cm lever arm.

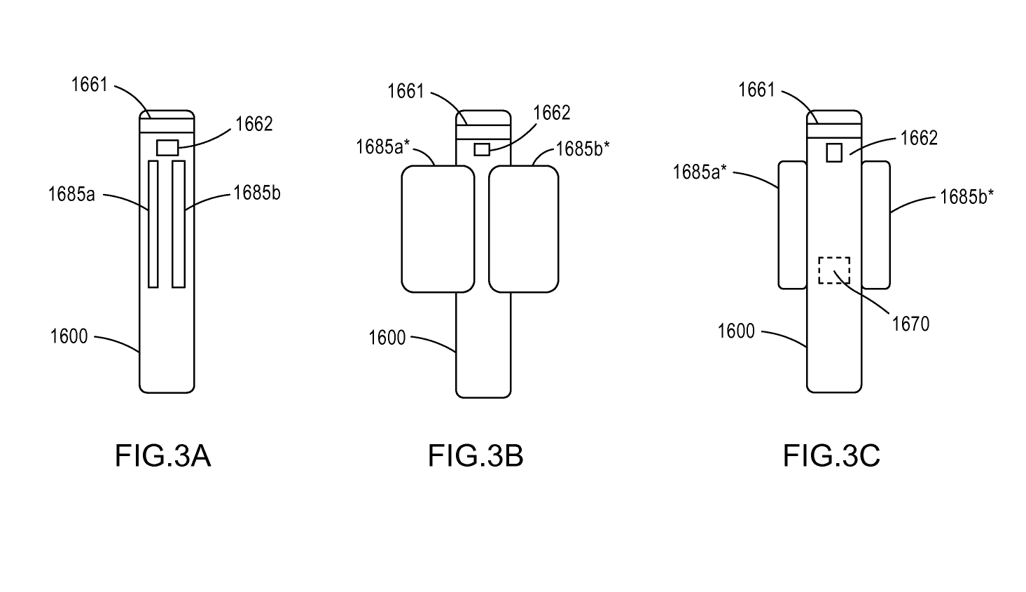

Typically, the support catheter 1600 includes an expansion member 1685 that presses the support catheter 1600 against the vessel wall such that the puncture element can enter the vessel wall in a controlled manner (as shown in FIGS. 1G-1I). Particularly, the expansion member 1685 presses the distal portion of the support catheter and the portion just proximal to the distal portion firmly against the vessel wall. The portion just proximal to the distal portion 1660 includes the exit port 1670. This region K is depicted in FIG. 1G. In certain embodiments, an expansion member is located on a side opposite of the exit port 1670. In embodiments, in which the catheter 1600 includes the flat surface 1640 of the distal portion 1660, the expansion member is located on a side opposite of the flat surface 1640 and exit port 1670. Alternatively, the support catheter 1600 may include two or more expansion members 1685 to press region K of the support catheter against a vessel wall. FIGS. 3A-3C depict an embodiment of the support catheter 1600 with two expansion members 1685a, 1685b. The expansion members 1685a, 1685b are in their non-expanded state in FIG. 3A. When expanded, the two expansion members 1685a*, 1685b* both act to firmly/securely press the catheter 1600 against the vessel wall with bi-pod support to reduce movement of the catheter during the procedure. The expansion members 1685a, 1685b may be an inflatable balloon that can be inflated via air or fluid. However, any expansion member is suitable for use in systems and methods of the invention. For example, the expansion member may include an expandable cage.

The distal portion 1660 must be stiff enough to resist bending in about any axis (by more than 2 mm over a 6 cm lever arm) along the entire length of the expanded expansion mechanism while the mechanism is expanded. For example, if a balloon is expanded causing even a curved vessel to straighten out and causing the vessel wall to conform along the distal portion of the support mechanism, the distal portion must be stiff enough to resist bending as a result of the tensioned wall, for the entire axial length of the expanded balloon.

FIGS. 1G-1I depict the puncture element 1680 deployed from the exit port 1670 of the support catheter 1660 at various modes of deployment. Specifically, FIGS. 1G-1I depict the dimensions of puncture height (D-ph) of the puncture element. The puncture height dictates how deep within the thickness of the vessel wall, the puncture element will enter and therefore, what plane a hydrodissection will create. In FIG. 1g, the puncture element 1680 exits the dissection probe in line with the flat support surface 1640 of the support structure. In this depiction, the puncture element can be advanced while sliding along the flat support surface 1640. In this embodiment, the diameter of the puncture element 1680 itself (if the bevel 1681 is oriented as shown), dictates the puncture element 1680 puncture height (D-ph). This embodiment represents the shallowest possible dissection plane within the vessel wall for a given puncture element 1680 diameter and at the depicted bevel 1681 orientation. In FIG. 1H, the mechanism is designed such that the puncture element or needle 1680 exits the dissection probe exit port 1670 parallel to the flat support surface of the support structure, but at a constant, non-zero height above the flat surface 1640. D-ph should be chosen to be smaller than the vessel wall thickness, such that when the puncture element 1680, which itself has a diameter that is necessarily smaller than the vessel wall thickness, is advanced into the wall, it cannot puncture through the outer side (the adventitia) of the vessel wall. In some embodiments an ideal puncture height is between 0.010″ and 0.100″. In some embodiments an ideal puncture height is between 0.015″ and 0.060″. In some embodiments an ideal puncture height is between 0.020″ and 0.040″. In some embodiments an ideal puncture height is between 0.025″ and 0.030″. FIG. 1I depicts a few other critical dimensions. The dimension D-off represents the distance between the flat support surface 1640 and the outermost edge 1682 of the support structure 1660. Upon inflation of the expansion mechanism 1685 (here a balloon), the vessel wall will conform to the outermost edge 1682 of the support structure proximal to the exit port 1670, and will conform to the flat supporting surface 1640 distal to the exit port 1670. Thus, D-off represents the amount of offset the two portions of vein wall will take. In some embodiments D-off is between 0.005″ and 0.060″. In some embodiments D-off is between 0.010″ and 0.040″. In some embodiments D-off is between 0.016″ and 0.030″. In some embodiments the support structure isn't flat but has a concave curvature. In other embodiments the support structure is not flat, but has a convex curvature. In both of these cases, the dimensions described here are in reference to the center-line of support surface, which will correspond to a minimum or maximum dimension.

FIG. 1I also depicts two other critical dimensions, proximal balloon length (D-bp) and distal balloon length D-bd). In the embodiment shown, a semi-compliant balloon 1685 (sometimes another type of expanding element) is expanded from the back side of the support structure 1688, which works to create a straight section of apposition between the support structure surface 1640 and the vessel wall. D-bp represents the distance the fully inflated balloon 1685 covers proximal to the exit port 1670, from which the puncture element 1680 or dissection probe 1683 emerges and punctures the vessel wall. D-bd represents the distance the fully inflated balloon 1685 covers distal to the exit port 1670. In some embodiments, vessel wall puncture will occur distal to the port 1670 itself. In these embodiments, these distances will be measured from the puncture site. In some embodiments, D-bp is chosen to be between 0 mm and 15 mm. In some embodiments, D-bp is chosen to be between 2 mm and 10 mm. In some embodiments, D-bp is chosen to be between 4 mm and 8 mm. In some embodiments, D-bd is chosen to be between 2 mm and 40 mm. In some embodiments, D-bd is chosen to be between 5 mm and 30 mm. In some embodiments, D-bd is chosen to be between 10 mm and 20 mm.

The puncture element 1680 may include an imaging element 1663, a functional measurement sensor 1664, an expandable member, or combination thereof. As shown in FIGS. 1G-1I, the puncture element 1680 includes a functional measurement sensor 1664 and an imaging element 1663. In certain embodiments, the imaging element 1663 allows one to obtain real time image of the vessel wall being penetrated by the puncture element 1680. In addition, the functional measurement sensor 1664 allows one to take functional measurements within the intraluminal space to determine, for example, whether the puncture element 1680 pierced through the vessel wall. For example, pressure or flow measurements would change within the intraluminal space if the puncture member broke completely through the vessel wall.

In certain embodiments, the puncture element 1680 is considered a dissection probe itself. In other embodiments, the puncture element 1680, depicted in FIG. 1F-1H, is a separate element that is moveably disposed within a dissection probe 1683. The dissection probe 1683 provides added support and stability to the puncture element, and prevents the puncture element from deviating from its desired path into the vessel wall during deployment. The dissection probe 1683 can be advanced over the puncture element 1680 and into the pocket after the puncture element has been sufficiently advanced. These embodiments depict an exit ramp 1686 and exit port 1670 that allow the probe 1683 to be advanced out of the tool lumen, while controlling the puncture height D-bh. The dissection probe 1683 may include an expandable member 1668, imaging element 1666, or functional measurement sensor 1667 (as shown in FIG. 1I). According to these embodiments, the dissection probe 1683 can be used to expand the space within the vessel wall, which was initially formed with the puncture element 1680. In addition, the dissection probe 1683 can be used to image and obtain functional measurements within the intramural space.

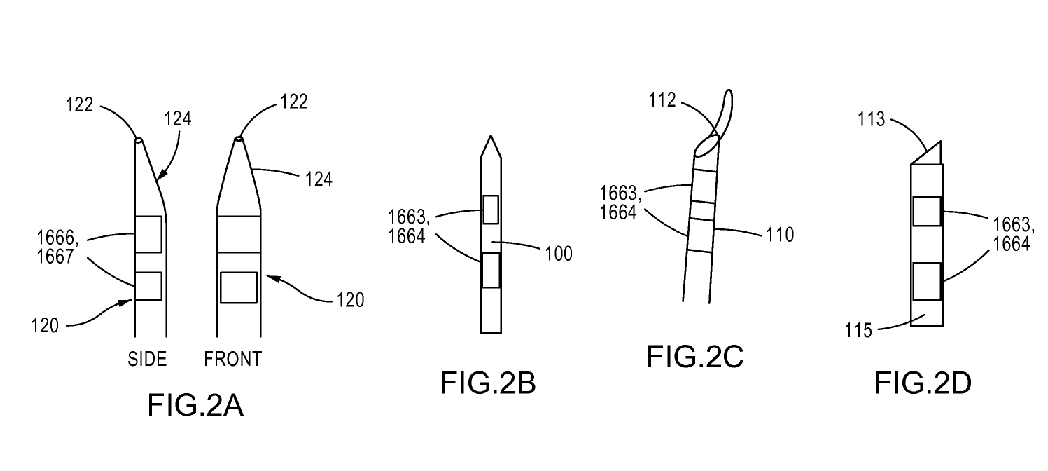

FIG. 2A depicts another embodiment of a dissection probe 120. The dissection probe 120 may include imaging element 1666 and/or functional element 1667. As shown in FIG. 2A, a dissecting probe 120 with radially asymmetric geometry is used. In this way, a puncture element protruding from the distal tip 122 will contact a vessel wall (even at a very shallow angle) prior to the full diameter of the probe proximal to the taper 124 (FIG. 2A). This radially asymmetric dissecting probe may be used in combination with a puncture element (such as a trocar device or other previously described embodiment). This combination of elements may itself be used in combination with all other embodiments previously described. For example, it may be used in combination with a support catheter of the invention with expansion elements, such that it is pushed out of an exit port toward or into a vessel wall. As shown in FIG. 2A, the dissection probe 120 also includes an imaging element and a functional measurement element. According to certain embodiments, the dissection probe 120 is configured to rotate. For example, the dissection probe 120 may be coupled to a rotary drive shaft. Rotary drive shafts, and rotational members disposed within catheters are known in the art. The rotation of the dissection probe 120 can serve two purposes. First, the rotation can act as a means to further separate the tissue layers of the vessel wall for intramural space formation. Second, the rotation can serve to assist in imaging the luminal surfaces of the vessel wall (e.g. the luminal surfaces within intramural space). For example, imaging elements (such as optical coherence tomography and ultrasound imaging elements) capture cross-sectional imaging data obtained during a rotation of the imaging element. Different types of imaging elements (rotational and non-rotational) are described hereinafter.

FIGS. 2B and 2C depict various distal portions of puncture elements. Any of the puncture elements may include a functional measurement element 1663 or an imaging element 1664 (as shown). The puncture elements are deployable from an exit port of a support catheter to form the intramural space within the vessel wall. Preferably, the puncture elements of the invention define a lumen with an opening at the distal tip through which a hydrodissection fluid can be ejected from. The hydrodissection fluid assists in separating the tissue layers to form the intramural space. As shown in FIG. 2B, the puncture element is a pencil point trocar device 100. This geometry may contain an internal lumen so that it may also be used in conjunction with subsequent hydrodissection through the probe lumen after puncture (FIG. 2B). The pencil point trocar device 100 may be used in combination with all other embodiments previously described. The pencil point trocar puncture element 100 may include an opening at its distal tip leading to a lumen for purposes of delivering hydrodissection fluid. In some embodiments, the puncture element is a skived puncture element 110 with a shovel like geometry 112. The shovel-like geometry 112 is used to help skive the vessel wall so that as thin a flap as possible is created in the vessel wall. A hollow lumen within this probe may then be used for hydrodissection after creating this wall defect, much like in other embodiments described (FIG. 2C). The skived puncture element 110 with shovel like geometry 112 may be used in combination with all other embodiments previously described. For example, it may be used in combination with a tubular assembly with expansion elements, such that it is pushed out of an exit port toward or into a vessel wall. FIG. 2D shows puncture element 115 with a beveled-tip 113. The puncture element 115 may include an opening at its beveled-tip 113 leading to a lumen for purposes of delivering hydrodissection fluid.

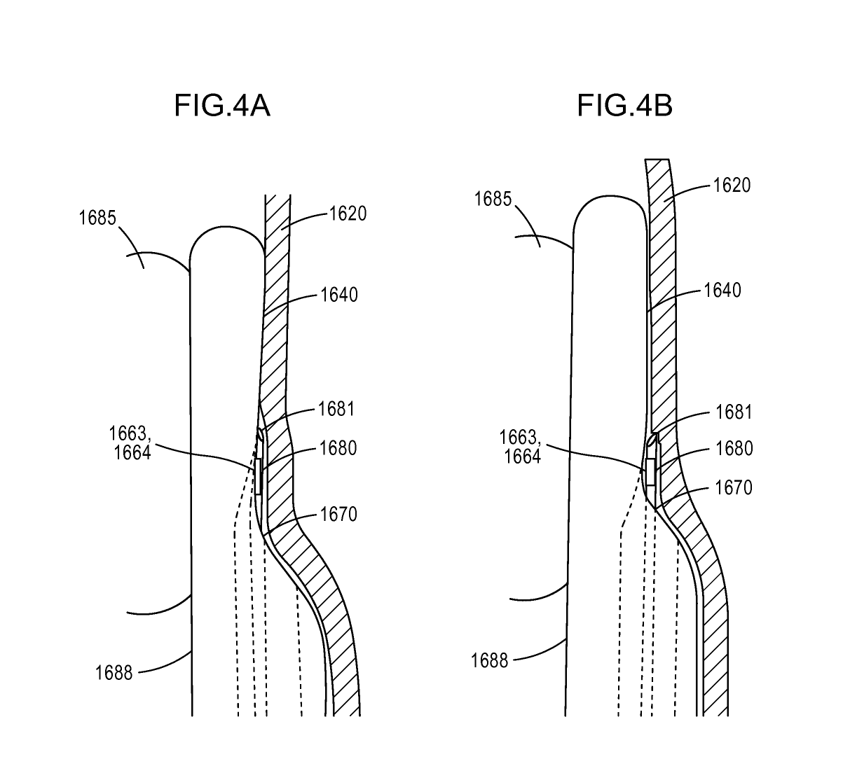

FIG. 4A and FIG. 4B describe a method for controllably entering the vessel wall 1620 with a puncture element 1680. As described in a previous embodiment, the puncture height of the puncture element is determined by the geometry of the flat surface 1640 of support catheter 1600, the puncture element 1680 diameter, and the angle of the bevel 1681 of the puncture element 1680 (here a beveled needle) with the vessel wall 1620. In the following embodiment, the user has the ability (active or passive) to rotate the puncture element 1680 about its longitudinal access, thus changing the bevel 1681 angle with respect to the vessel wall 1620, and thus changing the puncture height. In this embodiment, FIG. 4A depicts the expansion mechanism 1685 (a balloon or cage) having just been expanded off the opposing side 1688 of the support catheter, forcing the vessel wall into the flat surface 1640 of the support catheter 1600, while the puncture element 1680 is already in a starting position outside the exit port 1670 of the support structure, and therefore in contact with the vessel wall. The beginning angular orientation of the puncture element 1680 and bevel 1681 is such that the puncture height is minimized for the given puncture element diameter and outlet height (0°). FIG. 4B depicts a method for gaining controlled entry into the vessel wall 1620 without traveling all the way through the wall, by simply rotating the puncture element 1680 toward 180°, or an angular orientation that maximizes the puncture height for the given puncture element 1680 diameter and outlet height. The distal sharp tip or bevel 1681 of the puncture element 1680 is in this way inserted into the vessel wall 1620 due to the counter-tensions provided by the expansion element 1685 on the support catheter 1600. In a similar embodiment, this rotational entry method can be accomplished with slight forward advancement of the puncture element 1680 right after rotational bevel entry into the wall. In another similar embodiment, this rotational entry method can be accomplished with slight forward advancement of the puncture element 1680 during rotational bevel entry into the wall. Any of these methods can be employed by a mechanism that allows the user the ability to manually trigger rotational movement and translational movement (advancement) of the puncture element. In other embodiments, all of these methods can be employed by a mechanism that provides an automated combination of rotation and translation of the puncture element with a single trigger mechanism imparted by the user, such as a button, lever, or handle movement. While the puncture element 1680 is entering the vessel wall 1620 in both FIGS. 4A and 4B, an operator can receive real-time images or functional measurements from imaging element 1664 and imaging sensor 1664.

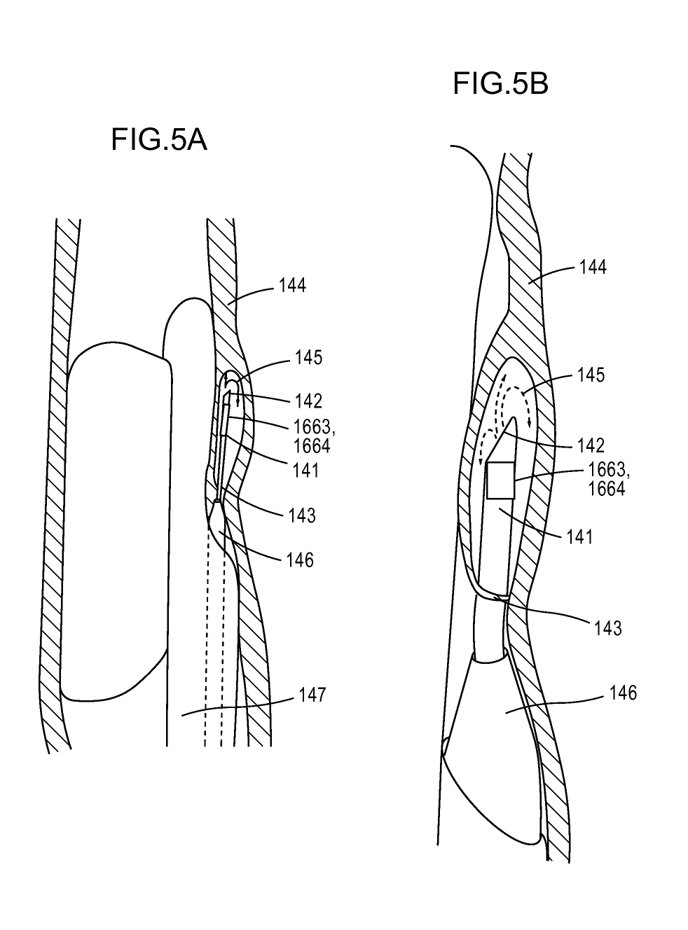

FIG. 5A and FIG. 5B depict a puncture element 141 deployed out the support catheter 147 and into the vessel wall 144. As shown, the puncture element 141 is also deployed out of a dissection probe 146, although it is understood that the dissection probe 146 is optional. A hydrodissection fluid 145 is ejected out of the distal tip of the puncture element 141 to create a space between tissue planes of the vessel wall, which ultimately can form a tissue flap. The puncture element 141 may include an imaging element 1663 and functional measurement element 1664 that allow an operator to receive real-time images or functional measurements during the dissection. For example, a change in pressure or flow within the intraluminal space could advantageously signal to the operator that a hole has accidently formed through the vessel wall. As further depicted in FIGS. 5A and 5B, the puncture element 141, which has a constant diameter proximal to the bevel at the distal tip 142, holds a sufficient seal along the inlet 143 during hydrodissection, and thus can be used as an initial probe to penetrate to a proper depth within the vessel wall 144 by being advanced while expelling a hydrodissection fluid 145. A valve creation mechanism (e.g. an expandable member on a dissection probe) can then be advanced over this puncture element when necessary 146.

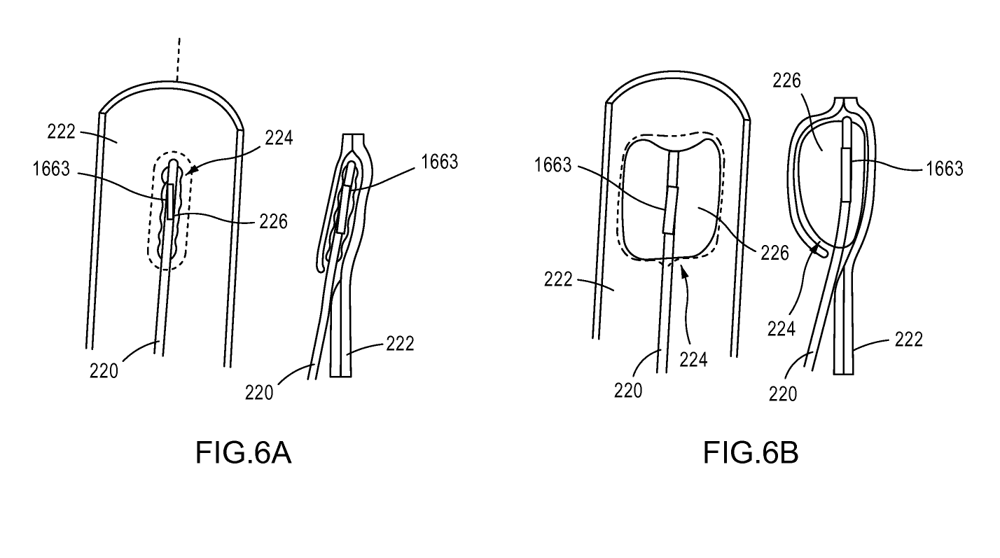

FIGS. 6A-6B depict a puncture element 220 with an expansion member that is used to form a pouch between tissue layers. Typically and as shown, the expansion member 224 is a compliant balloon 226. The puncture element 220 may also include an imaging element 1663 disposed beneath the expansion member. In such embodiments, the expansion member 224 is formed from a material that is transparent to the imaging modality of the imaging element 1663 (such that the imaging element can send and receive signals through the expansion member 224). As shown in FIGS. 6A-6B, a tissue dissection probe 220 (e.g. puncture element) is introduced into a vessel wall 222 some distance. Typically, the tissue dissection probe 220 is not pushed not entirely through the adventitia of the vessel. The probe 220 is then advanced within the vessel wall 222 distally (distal may be closer or farther from the heart depending on the direction of insertion), with the assistance of hydrodissection or manual blunt dissection. Once the tissue dissection mechanism 220 has been advanced to a sufficient depth, a expansion member 224 is actuated to expand and create a pouch of known geometry (FIG. 6B).

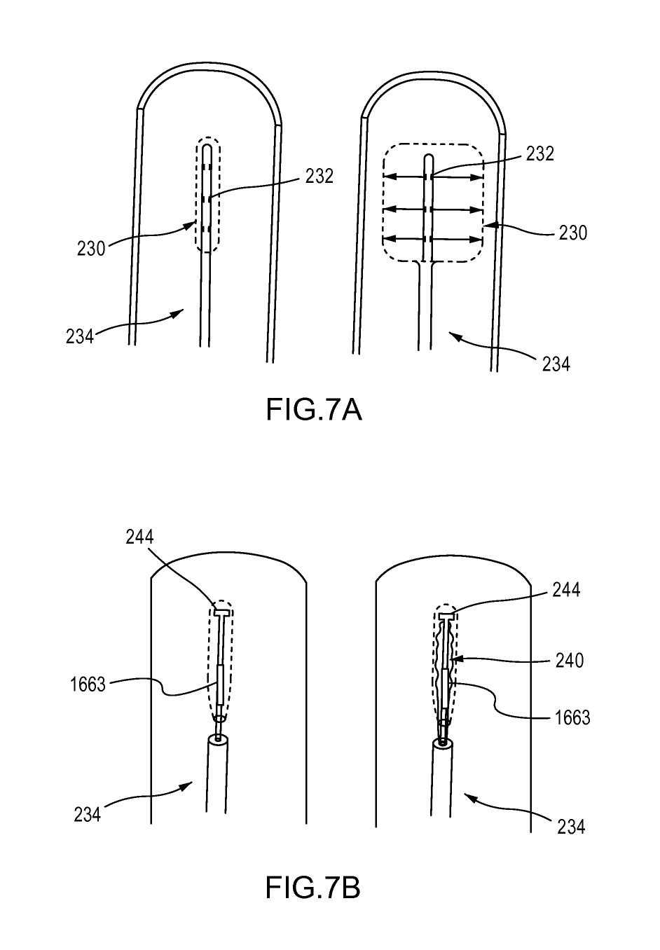

In other embodiments of this kind, a controlled hydrodissection mechanism 230 is used to create the pouch (see FIGS. 7A and 7B). This can be accomplished with a plurality of fluid ports 232 on the puncture element 234. As this embodiment, the puncture element 234 separates layers of the vessel wall, while simultaneously forming a pouch within the vessel wall. The puncture element 234 may be coupled to a mechanism to control fluid pressure and flow direction. In another embodiment, an expansion member 240 may be slideably moved over the puncture element 234 after the puncture element has entered the vessel wall (as depicted in FIG. 7B). The puncture element 234 may include a stopper 244 to prevent the expansion member 240 from falling off the puncture element 234. Once the expansion member 240 is in place, fluid can fill the expansion member 240 such that a pouch is formed in the vessel wall.

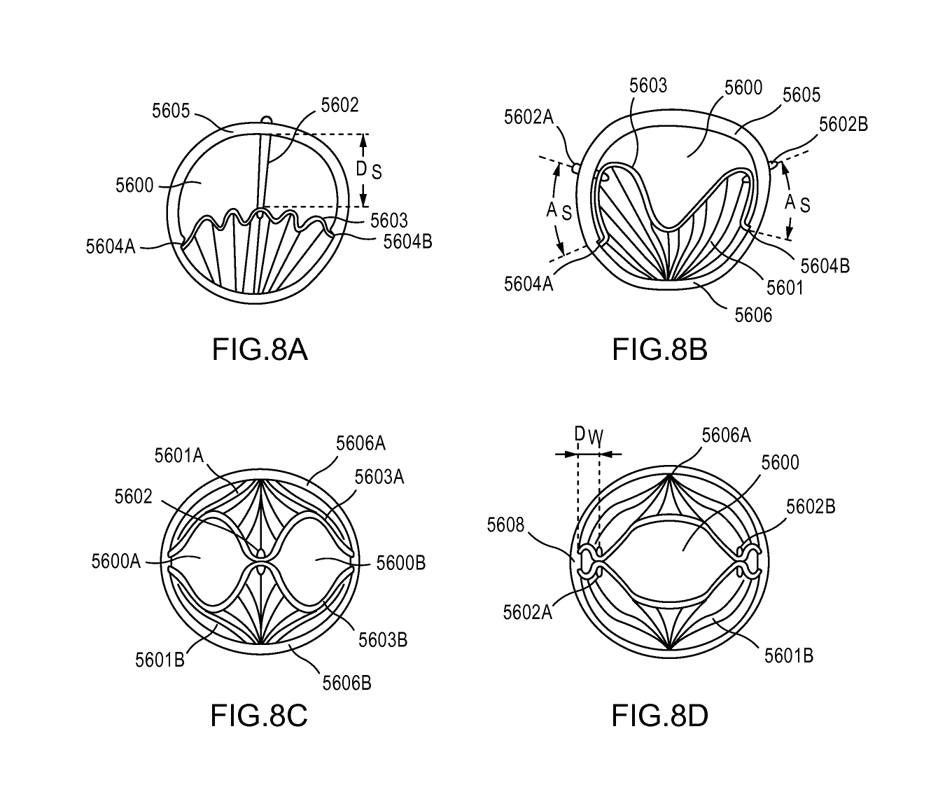

The larger the intramural space formed within the vessel wall, the larger the resulting tissue flap will be for forming an endoluminal valve within the vessel. After a valve pocket/valve flap has been created it is necessary to secure the valve flap to form the actual valve. Securing the valve flap also prevents it from re-adhering to the wall and, depending on the securement, controls hemodynamic properties associated with flow through the valve and the mechanics of the valve itself. FIGS. 8A and 8B depict stitching methods for monocuspid valves (greater than 180°), depicted in the open position, from a top down view, where the non-shaded region represents the true lumen 5600, and the shaded region represents the valve pockets 5601. FIG. 8A a depicts a method embodiment in which a stitch 5602 or other securement mechanism (such as a clip or a T tag) is placed at the center portion of the valve flap 5603 (equidistant from both edges 5604 a,b of the dissected flap 5603), and is connected on the other end to the fully thickness of the opposing vessel wall 5605. The stitch 5602 is maintained in a loose configuration (a long length before becoming taut), which allows blood to flow upward (out of the page) through the true lumen 5600, forcing the valve flap 5603 to open as much as is permitted by the stitch 5602. The stitch length (Ds) should be chosen to ensure the flap 5603 cannot re-adhere to the other vessel wall 5606 from which it first came. In some embodiments, the Ds should be between 20% and 95% of the vessel diameter. In some embodiments, the Ds should be between 50% and 90% of the vessel diameter. In some embodiments, the Ds should be between 70% and 80% of the vessel diameter. FIG. 8B depicts a different stitching method, which includes placing two stitches 5602 a, 5602 b, substantially symmetrically about the central axis of the vessel. In this embodiment, both stitches are placed a specific angle (As) from the edge of the tissue dissection flap 5604 a,b. In some embodiments, As is chosen to be between 5° and 80°. In some embodiments, As is chosen to be between 10° and 45°. In some embodiments, As is chosen to be between 15° and 30°. FIGS. 8C and 8D depict stitching methods for bicuspid autologous or natural valves. Valves are depicted in the open position, from a top down view, as blood is pumping upward (out of the page) through the true lumen 5600, to then later close the valves by flowing downward (into the page) into the valve pockets 5601. FIG. 8C depicts an embodiment in which a single tight stitch 5602 is placed along the center-line of the vessel lumen, bisecting each valve cusp 5603 a,b. This allows fluid to flow through two separate true lumen orifices 5600 a,b during the valve open phase. FIG. 8D depicts an embodiment in which two tight stitches 5602 a,b are placed symmetrically about the center-line of the vessel to permit only one major true lumen orifice 5600 for blood to flow through during the valve open phase. The stitches 5602 a,b are placed a certain distance from the vessel wall 5608 (Dw). In some embodiments, Dw is chosen to be between 1% and 40% of the vessel diameter. In some embodiments, Dw is chosen to be between 5% and 25% of the vessel diameter. In some embodiments, Dw is chosen to be between 10% and 20% of the vessel diameter.

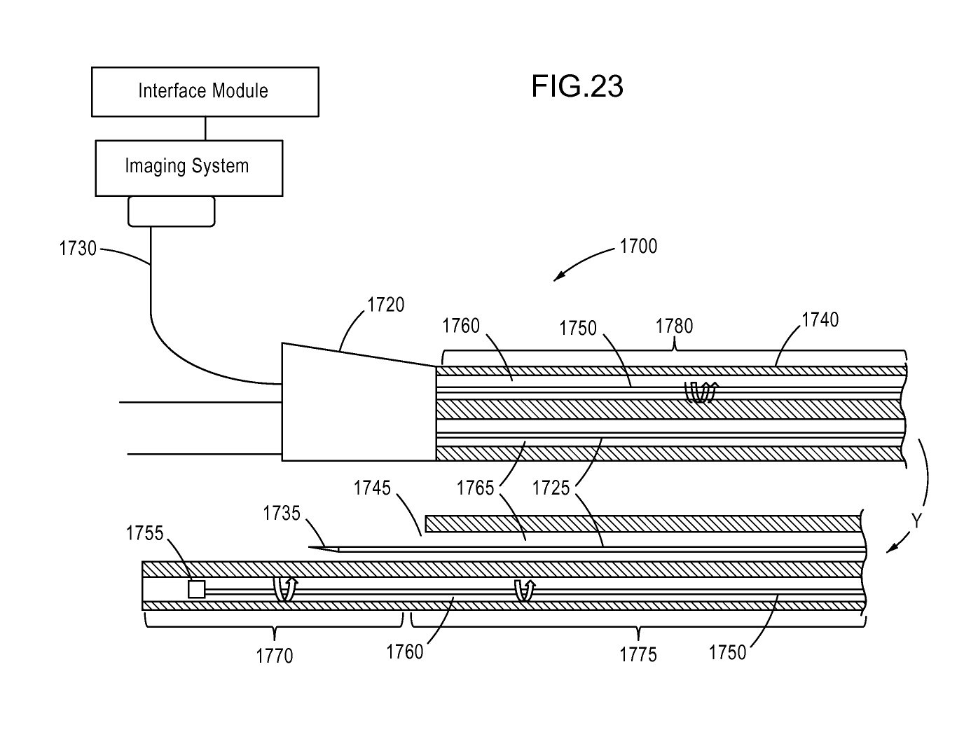

FIG. 23 depicts a schematic overview of valve-forming catheter system of the invention that includes a rotatable imaging element. The catheter system depicted in FIG. 23 is, for example, a variation of the catheter system depicted in FIGS. 1D and 1E. As shown in FIG. 23, the catheter system 1700 includes an elongate body 1740. The elongate body 1740 is segmented and curved (as shown by arrow Y) for purposes of illustrations. The elongate body 1740 includes a distal portion 1770, an intermediate portion 1775, and a proximal portion 1780. The elongate body includes two lumens 1760 and 1765. A rotary drive shaft 1750 coupled to a rotatable imaging element 1755 is deployed within the first lumen 1760, and a tissue dissection probe 1725 is disposed within the second lumen 1765. The tissue dissection probe 1725 is shown deployed through exit port 1745. The exit port 1745 is positioned such that the tissue dissection probe 1725 can be distally deployed in an orientation substantially parallel to the distal portion 1770 of the elongate body 1740. Although not shown in FIG. 23, the elongate body 1740 preferably includes one or more expandable elements that cause the distal portion 1770 and, in some cases, a portion of the intermediate portion 1775 against a vessel wall. This allows the tissue dissection probe 1750 to enter the vessel wall at such an angle that it forms a tissue flap within the intramural space without puncturing the vessel. The tissue dissection probe 1750 includes a tissue penetrating tip 1735 that defines an opening through which hydrodissection fluid may be deployed. The elongate body 1740 is coupled to a connector fitting 1720, which is attached to a proximal portion 1780 of the elongate body 1740. The connector fitting 1720 allows the signal lines 1730 of the imaging element 1755 to connect to the imaging system. The imaging system is connected to an interface module that allows an operator to receive real-time images of the vessel during formation of an intraluminal flap. If the catheter system 1700 also included a functional measurement sensor, the signal lines of the functional measurement sensor would also be able to connect to its system via the connector. The connector fitting may include or be connected to a fiber optic rotary joint in order to allow rotation of the signals lines distal to the rotary joint, while keeping the signal lines proximal to the rotary joint stationary without disrupting the power. Such rotary joints are known in the art.

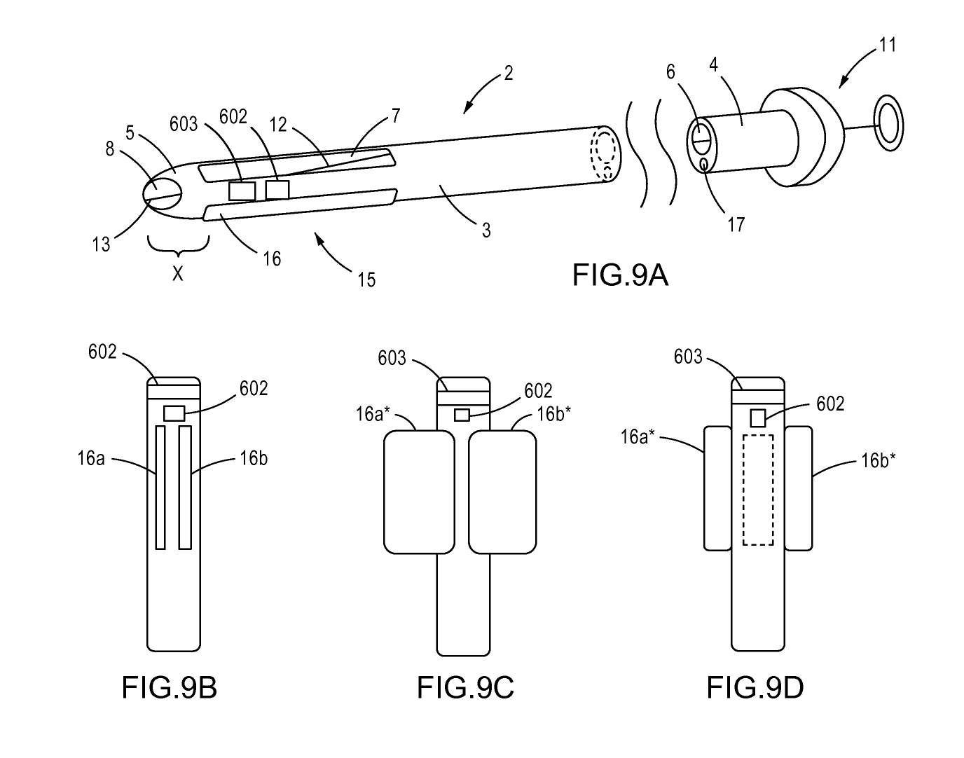

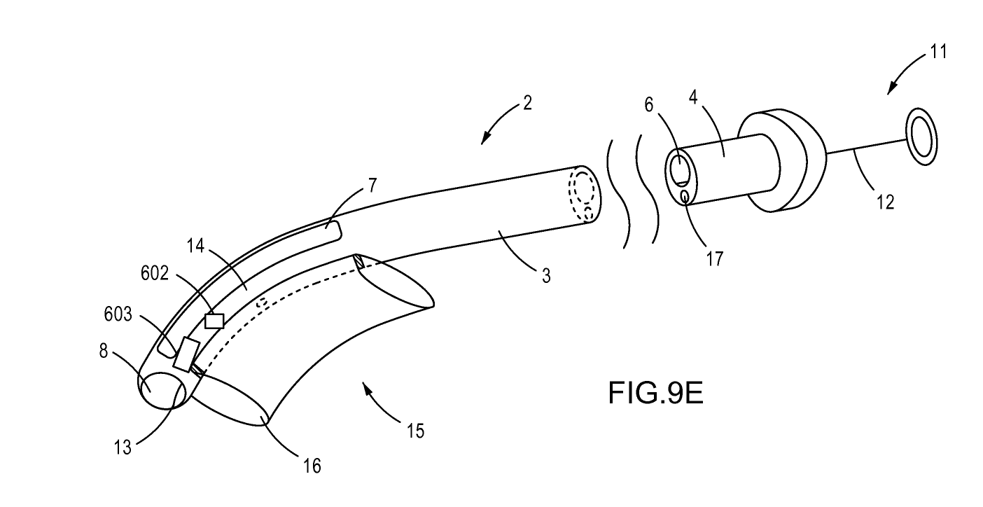

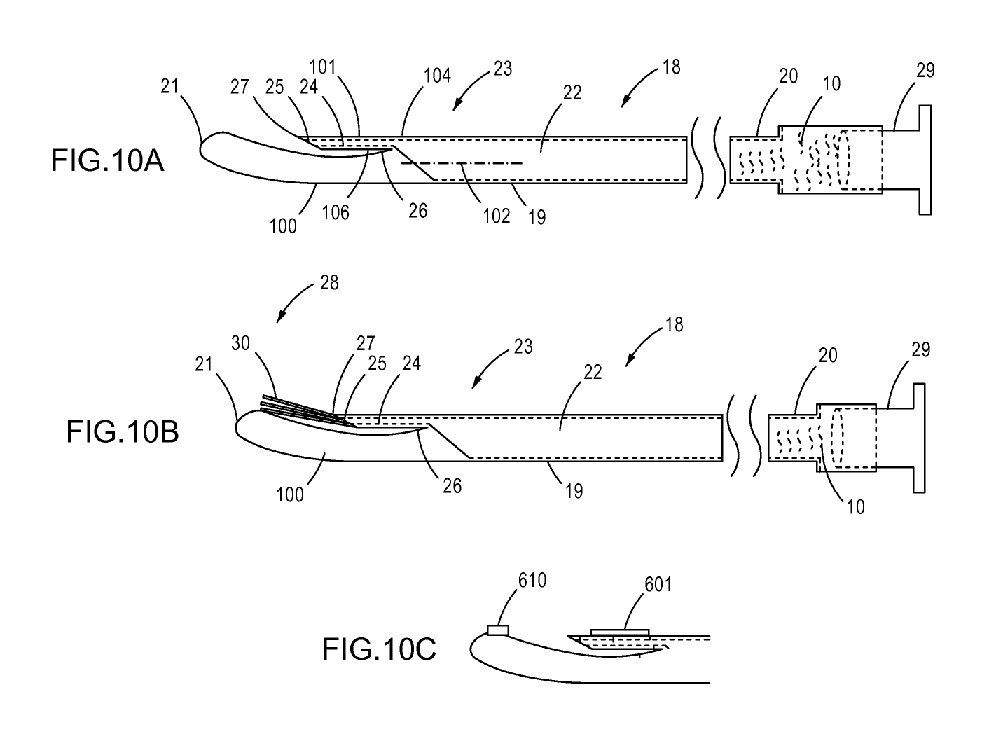

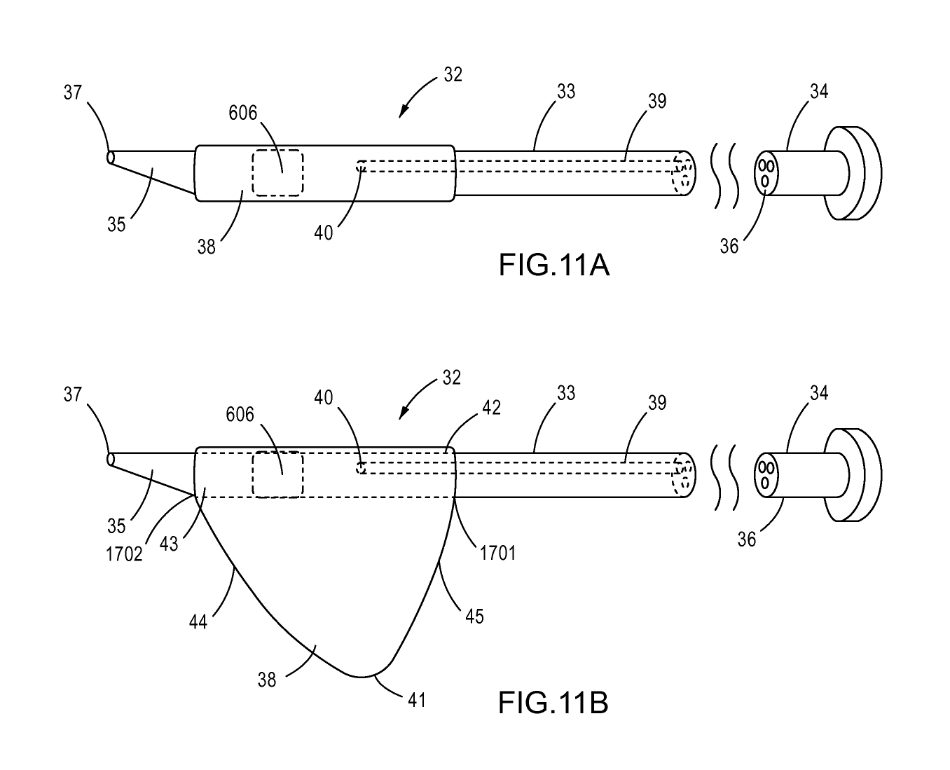

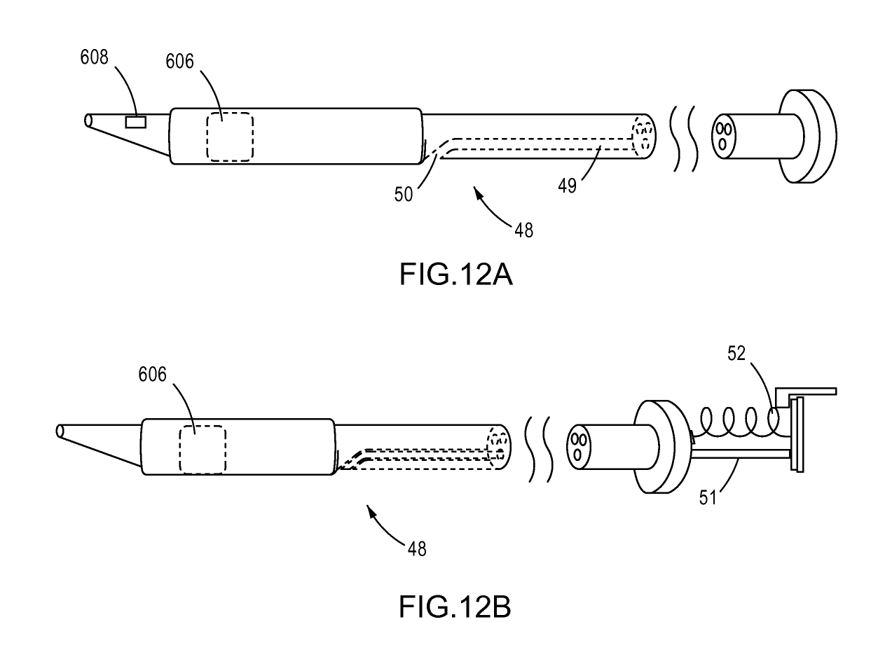

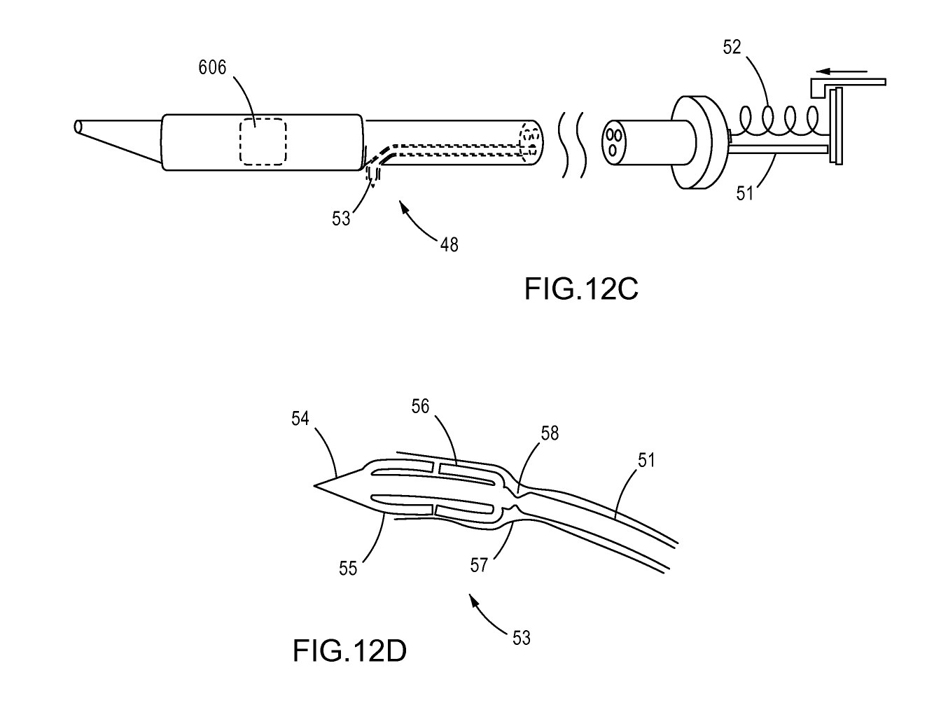

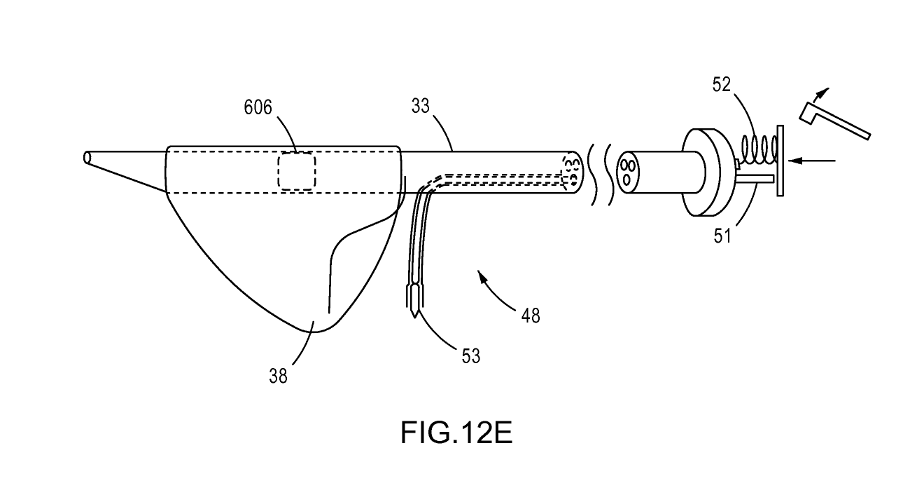

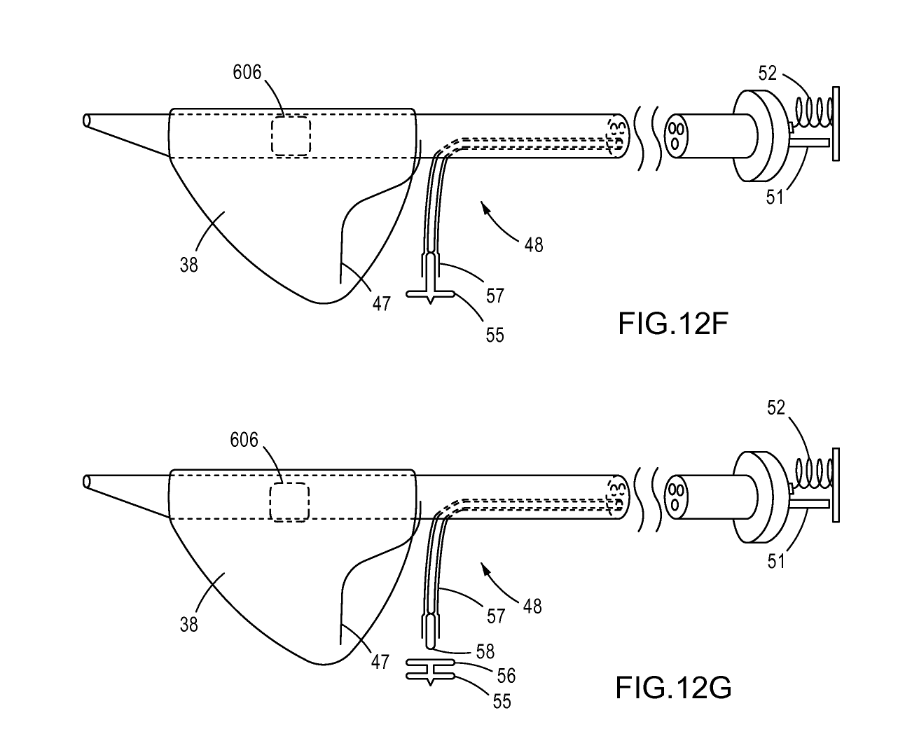

FIGS. 9A-12G depict another valve-forming catheter system of the invention according to certain embodiments. The valve-forming catheter includes a support catheter 2 (FIGS. 9A-9D), a sub-intimal access probe 18, (FIGS. 10A-10C), and a puncture element 48 (FIGS. 11A-12G). Each of the individual components of the valve-forming catheter system according to these embodiments are described individually first. After which, the support catheter 2, guide member 18, and tissue dissection probe 48 are shown and described as used together in the valve-forming catheter system in FIGS. 12A-12G.

FIG. 9A illustrates a support catheter 2 in accordance with some embodiments. The support catheter 2 includes an elongated tube 3 with a proximal end 4 and a distal end 5. The support catheter 2 has an internal lumen 6, which extends from the proximal end 4 to the distal end 5 of the elongated tube 3, terminating at a sideway facing exit port 7 near, but some small distance (e.g. 2 mm-10 mm) away from, the distal end 5 of the elongated tube 3. The support catheter 2 also includes a distal exit port 8 located at the distal most tip of the elongated tube 3, wherein the port 8 is in fluid communication with the internal lumen 6. The sideway facing exit port 7 is proximal to the distal portion X of the support catheter 2.

The support catheter 2 also includes an angling mechanism 11. In this embodiment, the angling mechanism 11 takes the form of a wire 12 connected with a mechanical bond 13 to the distal-most end of the internal lumen 6 of the conduit 2. In this embodiment, the angling mechanism 11 extends through the internal lumen 6 and past the proximal end 4 of the conduit 2. In this embodiment, the stiffness of the elongated tube 3 is lower at the distal end than at the proximal end so that when the wire 12 of the angling mechanism 11 is put into tension by the user at the proximal end, the elongated tube forms a curvature 14 near its distal end. Anyone skilled in the art of steerable catheters should understand how this mechanism can be used to create a curvature for the elongated tube 3. This curvature will allow tools to be passed through the sideway facing exit port 7 to take a non-parallel angle relative to the lumen wall, facilitating autologous valve creation. FIG. 9A depicts the support catheter 2 in a straight orientation before actuation of the angling mechanism 11, while FIG. 9E depicts the support catheter 2 in a curved orientation due to the actuation of the angling mechanism 11.

In the illustrated embodiments, the support catheter 2 also includes a wall-tensioning mechanism 15. As used in this specification, the term “wall-tensioning mechanism” or similar terms refer to any device that is configured to apply tension at a wall of a vessel. The wall-tensioning mechanism 15 includes a sideway-facing, inflatable, compliant balloon 16 of nearly cylindrical shape. The balloon 16 is coupled to the elongated tube 3 near the distal end 5 of the elongated tube 3. The balloon is in fluid communication with an inflation lumen 17, which communicates with an inflation port at the proximal end 4 of the elongated tube 3. The inflatable balloon 16 can be inflated to multiple diameters depending on the quantity and pressure of inflation fluid supplied through the inflation lumen 17. FIGS. 9A-9B depicts a non-actuated wall-tensioning mechanism 15 with a deflated expansion member 16 (such as a balloon), while FIG. 9C-9E depicts the wall-tensioning mechanism 15 in its actuated orientation with an inflated expansion member 16. The expansion member 16 is configured (e.g., sized, shaped, etc.) to be placed in a vessel. When expanded, the expansion member 16 applies a tension at the wall of the vessel.

As shown in FIGS. 9A and 9E, the wall-tensioning mechanism 15 includes only one expansion member 16. FIGS. 9B-9C depict an alternative embodiment in which the wall-tensioning mechanism 15 includes two expansion members 16a, 16b along a side of the support catheter 2 opposite of the sideways exit port 7. When expanded, the two expansion members 16a*, 16b* both act to firmly/securely press the catheter 2 against the vessel wall with bi-pod support to reduce movement of the catheter during the procedure. The expansion members 16a, 16b may be an inflatable balloon that can be inflated via air or fluid. However, any expansion member is suitable for use in systems and methods of the invention. For example, the expansion member may include an expandable cage.

As depicted in FIGS. 9A-9E, the support catheter 2 includes an imaging element 603 and a functional measurement sensor 602. While the support catheter 2 is shown with both the imaging element and functional measurement sensor, it is envisioned that the support catheter may include either one by itself. The imaging element allows one to image the luminal surface of the vessel in order to determine a certain location for the intramural space in the vessel wall for valve formation. The functional measurement sensor allows one to monitor vital signs within the vessel such as blood pressure and blood flow that also allow one to determine where valves are not properly regulating blood pressure and flow, and where a valve is needed to regulate pressure and blood flow.

FIG. 10A-10B depicts a sub-intimal access probe 18 in accordance with some embodiments. The sub-intimal access probe 18 may be used with the support catheter 2 of FIGS. 9A-9E. In particular, the sub-intimal access probe 18 may be introduced through the lumen 6 of the support catheter 2, and out of the sideway facing exit port 7.

In the illustrated embodiments, the sub-intimal access member 18 includes an elongated member 19 with a proximal end 20, a guide member 100 having a closed blunt distal end 21, an internal lumen 22, and a tissue engagement mechanism 23 extending from the elongated tube 19 at a location a small distance (e.g. 2 mm-8 mm) proximal to the closed blunt distal end 21. In this depiction, the tissue engagement mechanism 23 includes a tubular structure 101 with a lumen 24 in fluid communication with the main lumen 22 of the sub-intimal access member 18. There is therefore fluid communication from the proximal end 20 of the sub-intimal access member 18 through the entire length of the main lumen 22 of the sub-intimal access probe 18, into the lumen 24 of the tissue engagement mechanism 23, terminating distally at a forward facing exit port 25. In some embodiments, the tissue engagement mechanism 23 forms a relative angle with the elongated tube 19 of the sub-intimal access probe 18. The intersection of the tissue engagement mechanism 23 and the body of the elongated tube 19 creates a bottoming-out mechanism 26, in the form of an elbow joint. In some embodiments, the tissue engagement mechanism 23 may be attached to the elongated tube 19. For example, the tissue engagement mechanism 23 may be a part of the elongated tube 19. The tissue engagement mechanism 23 has a sharpened tip 27 at the distal end of the tubular structure 101, to facilitate penetration of an interior wall of a blood vessel. The sharpened tip 27 of the tubular structure 101 is proximal to the blunt end 21 of the guide member 100. The tubular structure 101 runs substantially parallel to the body of the guide member 100 such that a layer of skin tissue of the vessel wall fits between the guide member 100 and tubular structure 101 when the tubular structure 101 is deployed into a vessel wall. The angular orientation of the bevel of the sharpened tip 27 is such that the distal most point of the bevel is oriented furthest away from a longitudinal axis 102 of the sub-intimal access probe 18. In particular, the distal profile of the tip 27 tapers proximally from a first side 104 to a second side 106, wherein the first side 104 is further away from the axis 102 than the second side 106. Such configuration is advantageous because it allows the tip 27 to penetrate into the vessel wall more easily.

The sub-intimal access probe 18 also includes a tissue layer separation mechanism 28. As used in this specification, the term “tissue layer separation mechanism” or similar terms refer to any mechanism that is capable of separating tissue (e.g., dissecting tissue). The tissue layer separation mechanism 28 includes a pressurized source of fluoroscopic contrast agent 10, and a tissue layer separation actuator 29. FIG. 10A depicts the tissue layer separation mechanism 28 prior to actuation, at which point the pressurized source of fluoroscopic contrast agent 10 exists at the proximal end 20 of the sub-intimal access probe 18. FIG. 10B depicts the utilization of the tissue layer separation mechanism 28 during actuation, at which point the pressurized source of fluoroscopic contrast agent 10 is forced through the main lumen 22 of the sub-intimal access probe 18, and through the lumen 24 of the tissue engagement mechanism 23, until it exits out of the forward facing exit port 25 as a high pressurized stream 30. The tissue layer separation actuator 29 is a manually controlled piston mechanism or syringe. The stream of high-pressure fluid 30 can be used to separate layers of a wall of a vessel by forcing its way between tissue layers, creating a semi-controlled hydrodissection (not depicted here). In some embodiments, the bolus of high-pressure fluid that that is expelled into the inter-layer dissection plane in the vessel is sustained for 3-4 seconds. In the illustrated embodiments, the fluid stream 30 provides a fluid pressure inside the vessel wall that is sufficient to dissect tissue in the vessel wall, but insufficient to puncture through the wall of the vessel. The fluid stream 30 may have a fluid pressure anywhere from 100 mmhg to 1000 mmhg. Also, in some embodiments, the fluid stream 30 may be in pulses.

In some embodiments, the agent 10 may be a contrast agent, which may be imaged using an imaging device, such as a fluoroscopic device. This allows the position of the device 18 to be determined, and the fluid path of the agent 10 to be visualized during delivery of the agent 10. This also allows the progress of the separation of the tissue layers in the vessel to be monitored. The distal tip 21 of the guide member 100 is configured to be placed on a surface at an interior wall of the vessel to thereby guide the positioning (e.g., orientation) of the tip 27 relative to the vessel wall surface. In some cases, pressure may be applied to the vessel wall surface by pushing the blunt tip 21 distally, which will apply tension to the wall surface, and/or change an orientation of the wall surface—either or both of which will allow the tip 27 to more easily penetrate into the wall of the vessel.

The distal tip 21 of the guide member 100 is configured to be placed on a surface at an interior wall of the vessel to thereby guide the positioning (e.g., orientation) of the tip 27 relative to the vessel wall surface. In some cases, pressure may be applied to the vessel wall surface by pushing the blunt tip 21 distally, which will apply tension to the wall surface, and/or change an orientation of the wall surface—either or both of which will allow the tip 27 to more easily penetrate into the wall of the vessel.

FIG. 10C depicts the sub-luminal access member 18 with an imaging element and a functional measurement element. According to certain embodiments, the tubular structure 101 of the tissue engagement mechanism 23 includes imaging element 601 that allows one to obtain images of the luminal surfaces while the member 18 is inserted into vessel wall. A functional measurement sensor 610 may also be included on the member 18. As shown, the functional measurement sensor is placed on the blunt tip 21 of the guide member 100. However, it is contemplated that the functional measurement sensor can be placed elsewhere on the member 18, such as on the tubular structure 101.

In some embodiments, the tissue layer separation mechanism 28 is configured to dissect tissue in the wall of the vessel to create a pocket inside the wall of the vessel having a size that is sufficient to form a flap at the vessel wall. In such cases, the fluid stream 30 functions as a sub-intimal pocket probe. In other embodiments, the tissue layer separation mechanism 28 is configured to deliver the fluid stream 30 to create an initial lumen in the wall of the vessel, and another device may be placed in the lumen to expand the size of the lumen to create a pocket that is large enough to form a flap at the vessel wall.

FIGS. 11A-12G depict a device configured to enter a vessel wall after the tissue layer separation mechanism 28 of the sub-luminal access member 18 creates the initial entry lumen within the vessel wall.

FIG. 11A depicts a sub-intimal pocket probe 32 in accordance with some embodiments. The sub-intimal pocket probe 32 has an elongated member 33, with a proximal end 34, a blunt, tapered distal end 35, and a contrast lumen 36, which extends from the proximal end 34 to a contrast exit port 37 at the distal end 35 of the mechanism 32. The sub-intimal pocket probe 32 also includes an inflatable, compliant pocket creation balloon 38, a balloon inflation lumen 39, and an inflation port 40, which connects the balloon inflation lumen 39 to the pocket creation balloon 38. In the illustrated embodiments, the pocket creation balloon 38 is bonded to the outer surface 33 of the sub-intimal pocket probe 32 to form an air-tight seal.

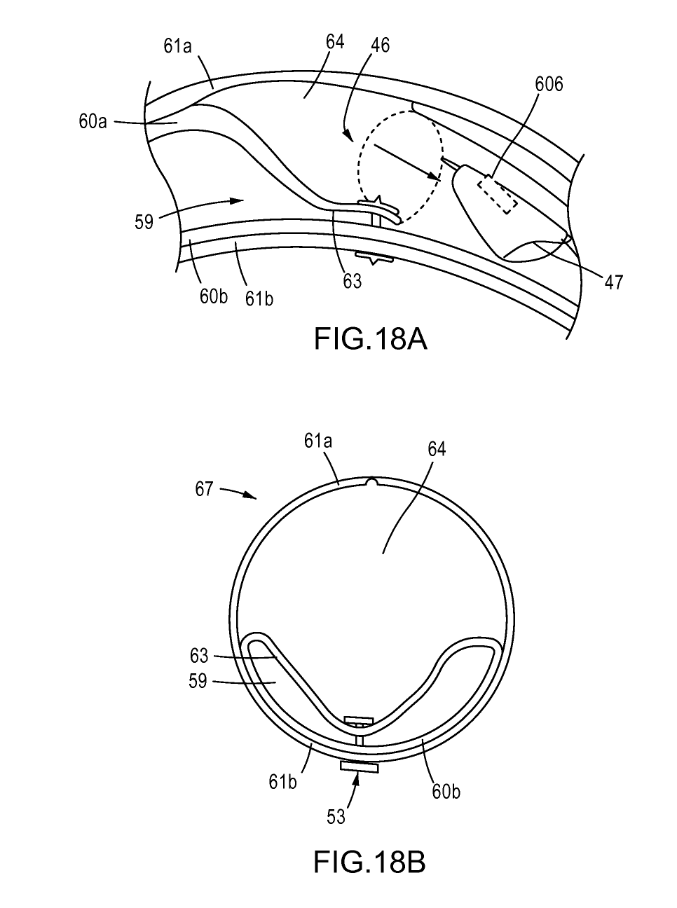

FIG. 11B depicts a configuration of the probe 32, in which the pocket creation balloon 38 is inflated. In the illustrated embodiments, the inflated balloon 38 takes an asymmetric shape upon inflation through the inflation lumen 39, which inflates sideways off of the outer surface of the sub-intimal pocket probe 32. The pocket creation balloon's largest diameter 41 is some distance closer to the proximal end 42 of the balloon than to the distal end 43 of the balloon. The balloon has a curved distal taper 44 and a curved proximal taper 45, the proximal one being more abrupt. In this embodiment, the sub-intimal pocket mechanism 32 is sized appropriately in its deflated orientation such that it has dimensional clearance through the main lumen 22 of the sub-intimal access probe 18, the narrow lumen 24 of the tissue engagement mechanism 23, as well as the forward facing exit port 25. As shown in FIGS. 12F and 12G, the balloon 28 may include a cutting element 47 configured to cut tissue to form a flap after pocket formation. The cutting of tissue in this manner is described in more detail with regard to FIGS. 18A-18B.

According to certain embodiments and as shown at least in FIGS. 11A and 11B, the pocket creation probe 32 includes an imaging element 606. The imaging element may be disposed on the surface of balloon 28 or on the surface of the pocket creation probe 32 beneath the balloon 28. When the imaging element 606 is beneath the balloon 28, the balloon 28 is formed from a material that is transparent to the imaging modality of the imaging element 606 (such that the imaging element can send and receive signals through the balloon 28). In addition to the imaging element, the pocket creation probe 32 may also include a functional measurement sensor 608. The imaging element 606 and functional measurement sensor 608 allow an operator to receive real-time images or functional measurements during the dissection and/or pocket creation. For example, a change in pressure or flow within the intramural space could advantageously signal to the operator that a hole has accidently formed through the vessel wall.

As shown in 11B, the balloon 28 includes a first end 1701 and a second end 1702. When expanded, the first end 1701 defines a larger volume than the second end 1702. With this configuration, the balloon 28 forms a cone or triangle shape. In such configurations, the balloon 28 may define a conical volume. The volume of the balloon 28 at the first end 1701 is larger than the volume at the second end 1701 in order to create a gradual tissue flap. The gradual tissue flap prevents excessive tension where the tissue flap merges with the vessel wall.

In some embodiments, the sub-intimal pocket mechanism 32 may optionally further include a channel for delivering a valve securement mechanism, wherein the valve securement mechanism is configured to secure a flap against a wall of a vessel. FIG. 12A illustrates a channel 49 located within the elongated member 33 of the mechanism 32, which is for delivering a valve securement mechanism. FIG. 12B depicts a valve securement mechanism 48 in accordance with some embodiments, particularly showing the valve securement mechanism 48 being delivered inside the channel 49. FIG. 12D depicts a more detailed view of the distal end 53 of the securement mechanism 48, which is comprised of a sharp puncturing member 54 at the leading end, two nitinol distal clip arms 55, two nitinol proximal clip arms 56, a constraining sheath 57, and a detachment joint 58, which is located at the interface between the securement delivery system 51 and the securement mechanism distal tip 53. In this depiction, the detachment joint 58 is shown as a notch in the wire. Returning to FIG. 12B, which depicts the securement delivery system 51 as a wire, and an actuation mechanism 52, depicted as a spring and latch system. In the illustrated embodiments, the channel 49 extends from the proximal end of the sub-intimal pocket probe 32 to an angled side port 50, through which valve securement will be accomplished. FIG. 12C depicts the valve securement mechanism 48 in its initial stage of deployment, in which the delivery system 51 has moved forward pushing the securement mechanism distal tip 53 out of the angled side port 50 by a short distance. FIG. 12E depicts the valve securement mechanism 48 in its second stage of actuation, as a result of activation of the actuation mechanism 52. In this embodiment, the activation of the actuation mechanism 52 occurs after inflation of the pocket creation balloon 38. The delivery system has moved forward to its maximum distance, pushing the securement mechanism distal tip 53 to a distance from the elongated member 33 slightly exceeding that of the outer most portion of the inflated pocket creation balloon 38. FIG. 12F depicts the valve securement mechanism 48 in its third stage of actuation, in an orientation in which the constraining sheath 57 has been retracted enough to allow the distal clip arms 55 to spring outward into an orientation perpendicular to the axis of the delivery system 51 as a result of their shape memory characteristics. The forth stage of actuation is accomplished when the constraining sheath 57 is retracted further to allow the proximal clip arms 56 to spring outward into an orientation perpendicular to the axis of the delivery system 51 as a result of their shape memory characteristics. FIG. 12G depicts the valve securement mechanism 48 in its fifth and final stage of actuation. After the valve securement mechanism 48 has been deployed to secure a flap against a vessel wall, the entire securement mechanism delivery system 51 is retracted forcing the securement mechanism distal tip 53 to detach from the securement mechanism delivery system 51 at the detachment joint 58. The detachment joint 58 is intentionally built to fail in tension at that location, so that the securement mechanism distal tip 53 is left behind upon retraction of the securement mechanism delivery system 51. In this embodiment, the securement mechanism distal tip 53 takes the final orientation of an “H-tag”. In other embodiments, the securement mechanism distal tip 53 may have other configurations (e.g., shapes). For example, in other embodiments, the securement mechanism may include one or more tines having different deployed shapes. Also, in other embodiments, instead of the above configurations, the securement mechanism 48 may be tissue glue that is deployed out of the channel 49, or another channel that is at a different device. The tissue glue is used to secure a flap against a vessel wall.

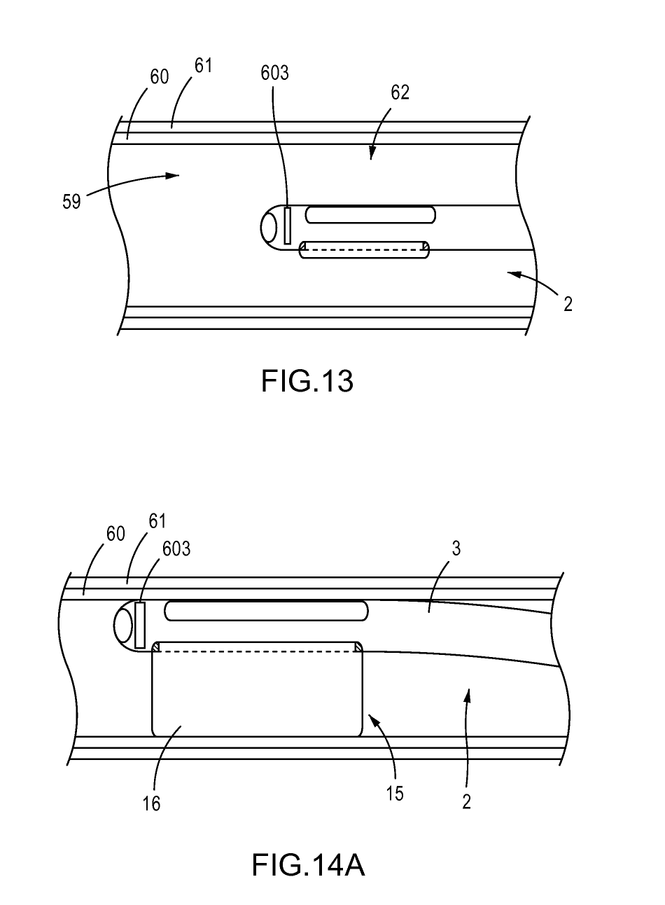

FIG. 13-FIG. 18B depict a method of using the devices depicted in FIGS. 9A-12G within the context of a percutaneous valve creation procedure. The described functionality is by no means intended to be descriptive of all possible uses of the devices. It should be noted that one or more acts/functionalities may be omitted for certain procedural situations.

FIGS. 13-18B portray the devices being used within a bodily lumen 59 of a vessel. For simplicity, the bodily lumen is shown with an inner layer 60, and an outer layer 61. In many bodily lumens, such as the vein, the lumen wall is composed of three layers: the intima, media, and adventitia. In the following representations, the inner layer 60 may represent the intima and the media combined, while the outer layer 61 may represent the adventitia. Alternatively, in some embodiments of valve creation, the inner layer 60 may represent the intima, while the outer layer 61 may represent the media and the adventitia combined. In still further embodiments, both the inner layer 60 and the outer layer 61 may include the media.

FIG. 13 depicts the support catheter 2 of FIG. 9A, which has been inserted percutaneously and delivered to the valve creation site 62 within a bodily lumen 59, from the retrograde direction. In some embodiments, a user of the device can receive real-time images of the support catheter moving through the vessel using imaging element 603. With the imaging element, the user may obtain cross-sectional images of the luminal surface, and allow the user to determine an ideal position within the vessel for flap formation. In addition to or alternatively, the user of the device may inject a fluoroscopic contrast agent 10 through the distal exit port of the support catheter 8, so that fluoroscopic visualization may be utilized to view the support catheter 2. This may allow the user to determine the position of the support catheter 2 relative to the valve creation site 62 in the vasculature.

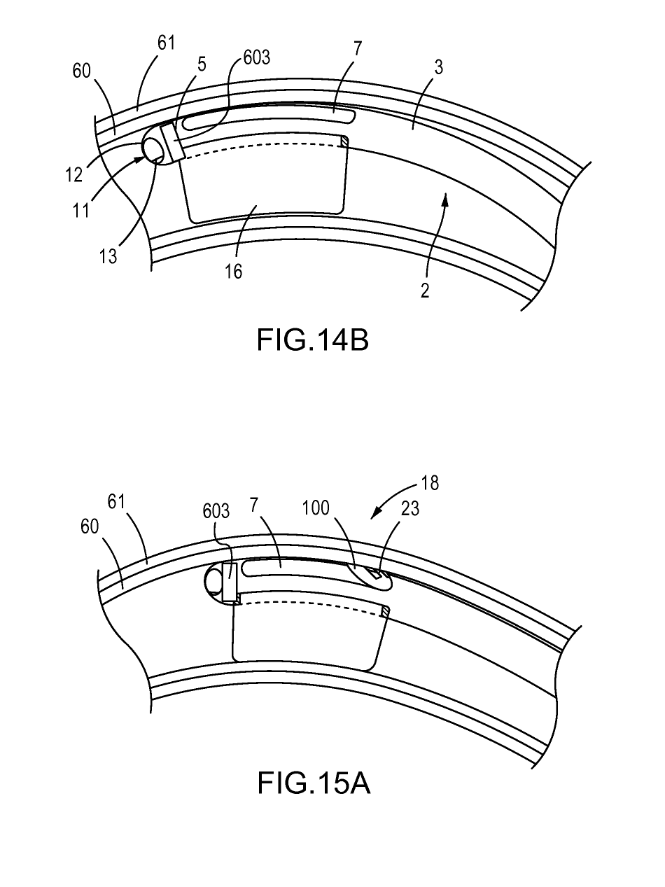

FIG. 14A depicts the support catheter 2, in which the wall-tensioning mechanism 15 has been actuated. In this depiction, the main functional component of the wall-tensioning mechanism 15 is an inflatable compliant balloon 16, which extends perpendicularly from the surface 3 of the support catheter 2 to the inner wall 60 of the bodily lumen 59. The balloon is inflated through the inflation lumen 17 incrementally until a particular pressure is measured which corresponds with proper lumen wall dilation.

FIG. 14B depicts the support catheter 2, in which the angling mechanism 11 has been actuated. In this depiction, the main functional component of the angling mechanism 11 is a wire 12, which is attached to a mechanical bond 13 to the distal-most end of the internal lumen 6 of the support catheter 2. In this depiction, the wire 12 has been tensioned from the proximal end, which forces the distal end 5 of the support catheter 2, into a bent orientation. With the wall-tensioning mechanism 15 actuated, the catheter surface 3 and the inflated balloon 16 are in flush contact with the inner lumen wall 60, and thus transfer their curved orientation to the bodily lumen 59 itself. In this way, the angling mechanism 11, forces the wall of the vessel to bend. In the illustrated embodiments, the majority of the curvature of the support catheter 2 occurs at or distal to the sideways facing exit port 7. This configuration is advantageous because it allows a tool passing out of the sideways facing exit port 7 to form a non-parallel angle with the wall of the vessel.

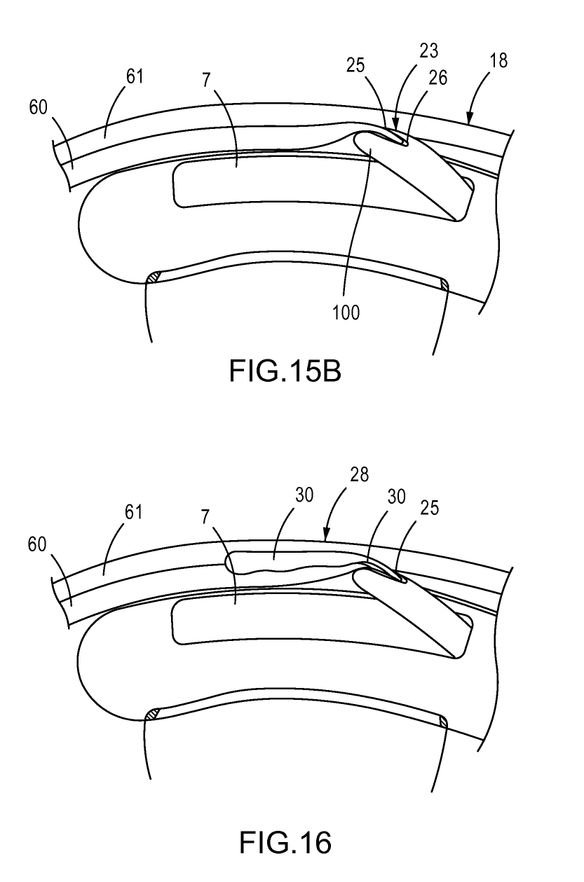

FIGS. 15A-B depict the sub-intimal access probe 18 located in the support catheter 2, and being deployed therefrom. FIG. 15A depicts the sub-intimal access probe 18 during actuation as it exits the sideways facing exit port 7, as a result of advancement from the proximal end of the conduit 4. Due to the curvature of the conduit distal to the sideways facing exit port 7, the sub-intimal access probe 18 exits the conduit at a non-parallel angle relative to the inner lumen wall 60. The guide member 100 is pressed against the vessel surface to guide the positioning of the tissue engagement mechanism 23. For example, the mechanism 23 may be tilted about the contact point between the guide member 100 and the vessel wall. Thus, the guide member 100 allows the mechanism 23 to enter the vessel wall at a desired angle. In some cases, the guide member 100 also provides some tension at the vessel wall surface (i.e., in addition to that already provided by the balloon 16). FIG. 15B depicts the sub-intimal access probe 18 after it has been advanced fully and has engaged the inner lumen wall 60. In the illustrated embodiments, the tissue engagement mechanism 23 penetrates the vessel wall, and is advanced until vessel tissue abuts against a stopper (e.g., the region where the proximal end of the tissue engagement mechanism 23 meets the guide member 100). Full engagement occurs after the tissue engagement mechanism 23 penetrates the vessel wall, and when the tissue between the guide member 100 and the mechanism 23 meets the elbow joint of the bottoming-out mechanism 26 (the stopper). Upon full tissue engagement, the forward facing exit port 25 of the tissue engagement mechanism 23 rests completely within the lumen wall.

FIG. 16 depicts the tissue layer separation mechanism 28 being used at the valve creation site 62. After the tip 27 of the tissue engagement mechanism 23 has been placed inside the wall of the vessel, the pressurized source of fluoroscopic contrast agent 10 is forced through the main lumen 22 of the sub-intimal access probe 18, and through the narrow lumen 24 of the tissue engagement mechanism 23, until it exits out of the forward facing exit port 25 as a high pressure stream 30. This stream of high-pressure fluid 30 acts to atraumatically separate the inner layer 60 from the outer layer 61 of the bodily lumen 59 at the valve creation site 62 by physically breaking interlayer bonds upon injection, creating a semi-controlled, inter-layer dissection plane 31. In some embodiments, the pressure of the stream 30 is sustained until the dissection plane 31 with a certain length has been created. In other embodiments, the stream 30 may be delivered in pulses. Also, in other embodiments, the pressure of the stream 30 may be adjusted (e.g., increased) as the length of the dissection plane 31 is increasing in size. High-pressure fluid dissection offers advantages over blunt mechanical dissection with a stiff probe. The dissection force imparted within the vessel wall is spread out over the internal surface area of the dissection pocket, and thus imparts less force in any one location than would a solid probe (or a solid device). Additionally, with fluid dissection, tissue separation automatically occurs along a plane of least-resistance, which may allow dissection to take place at a lower pressure (e.g., compared to using a solid device).

Because a fluoroscopic contrast agent 10 is used in tissue layer separation in this embodiment, the user will have the opportunity to visualize the effect of the fluid delivery on the tissue using fluoroscopic visualization techniques. In particular, through fluoroscopic visualization technique, the user may view the progress of the tissue dissection within the wall of the vessel. The fluoroscopic visualization technique also allows a user to determine if the dissection plane 31 is getting too close to the exterior surface of the vessel wall. In such cases, the user may determine that there is a potential that the vessel wall may be punctured (by the fluid) therethrough, and may stop the process. Additionally, this visualization technique allows the user to evaluate the depth and shape of the newly created inter-layer plane 31 to determine if the tissue layer separation mechanism 28 needs to be actuated again. This process may be repeated indefinitely until a proper tissue layer separation has occurred, which allows for continuation of the procedure.

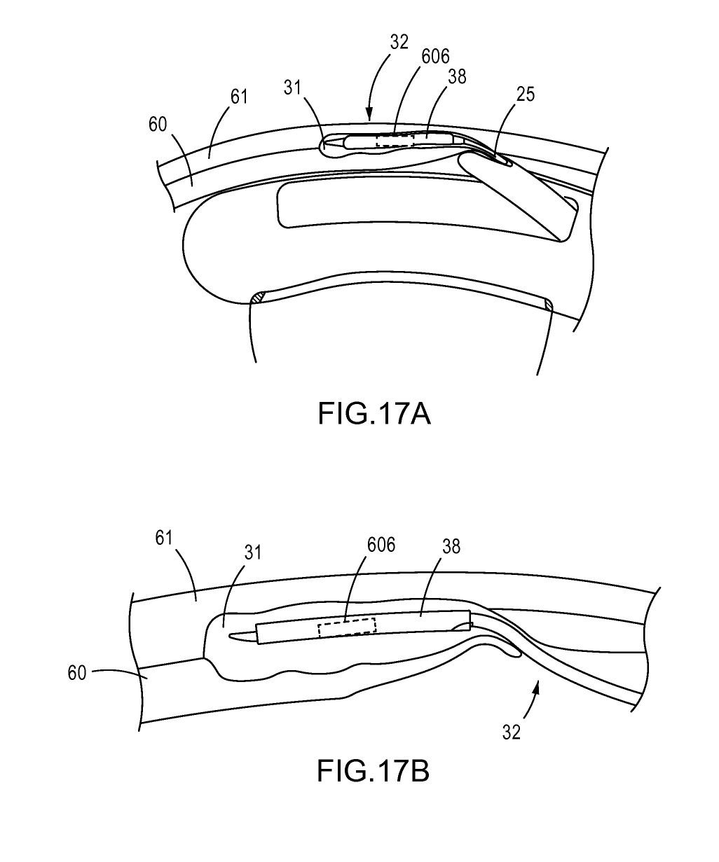

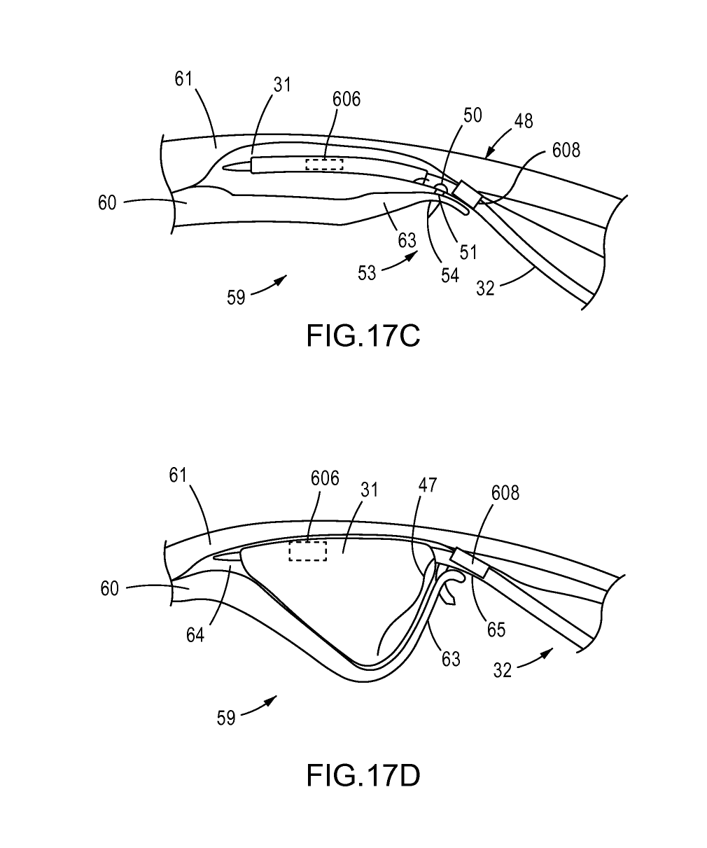

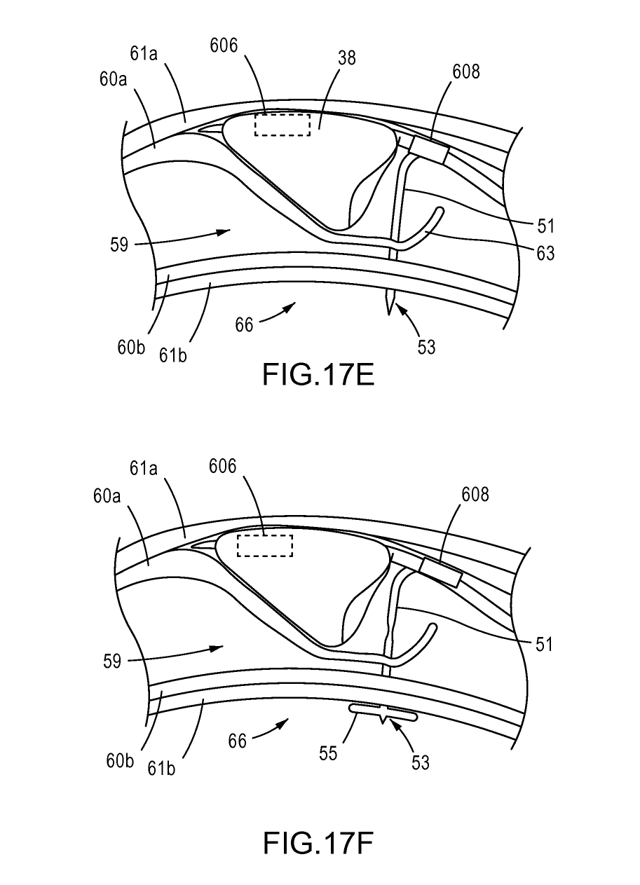

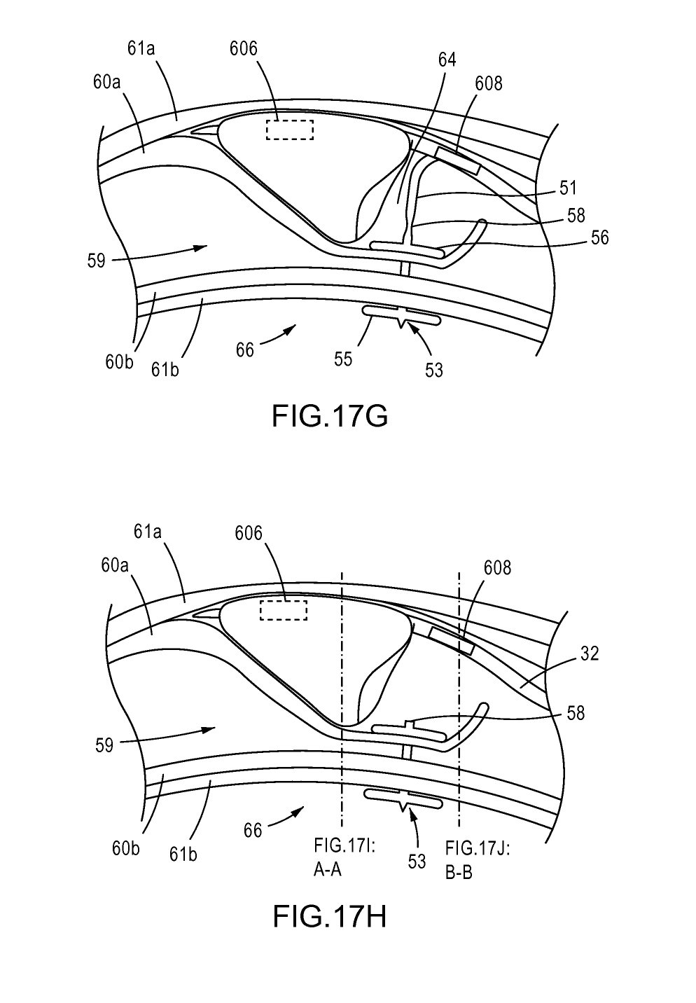

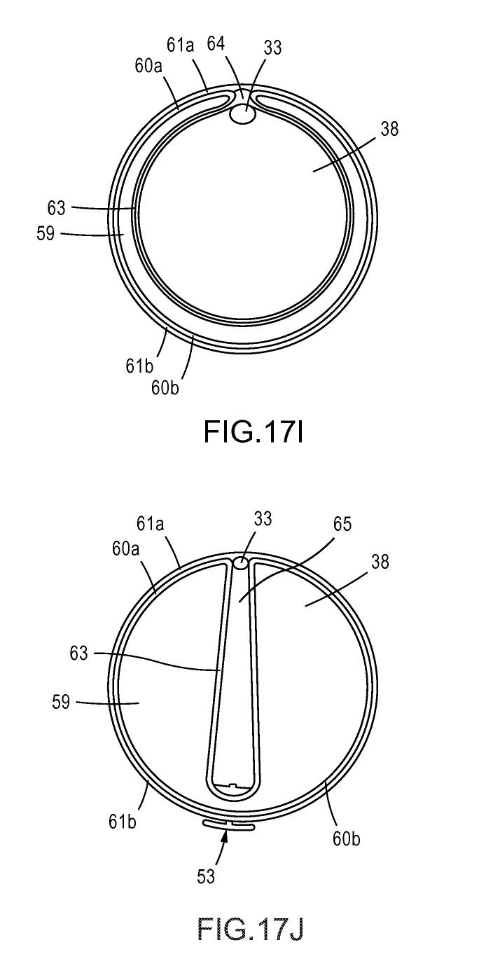

FIG. 17A depicts that the sub-intimal pocket probe 32 is advanced into the inter-layer plane 31. Following proper separation of tissue layers using the tissue layer separation mechanism 28, the sub-intimal pocket probe 32 is advanced through the main lumen 22 of the sub-intimal access probe 18, into the narrow lumen 24 of the tissue engagement mechanism 23, and out of the forward facing exit port 25. As depicted in FIG. 17A, the sub-intimal pocket probe 32 is advanced out of the forward facing exit port 25 of the tissue engagement mechanism 23, and into the newly created inter-layer plane 31 that now exists between the inner layer 60 and the outer layer 61 of the lumen wall. The sub-intimal pocket probe 32 is advanced far enough such that the proximal most portion of the deflated pocket creation balloon 38 is at least within the inter-layer plane 31. As shown, the pocket probe 32 includes imaging element 606, which allows a user to image the luminal surfaces within the vessel wall during the procedure.

FIG. 17B depicts that the sub-intimal access probe 18 along with the support catheter 2 has been removed, leaving only the sub-intimal pocket probe 32 behind, within the inter layer plane 31 previously created.

FIG. 17C depicts the first stage of actuation of the valve securement mechanism 48, which occurs prior to the sub-intimal pocket creation. Once the sub-intimal pocket probe 32 is advanced sufficiently into the newly created inter-layer plane 31, the securement mechanism delivery system 51 is advanced forward a small amount pushing the securement mechanism distal tip 53 out of the angled side port 50. Because of its sharp puncturing member 54, and the position and angular orientation of the angled side port 50 with respect to the newly separated inner tissue flap 63, the securement mechanism distal tip 53 punctures through the inner tissue layer flap 63 from its inter-layer plane 31 side, and emerges into the inside of the bodily lumen 59. The valve securement mechanism maintains control of the inner tissue layer flap 63 throughout the completion of sub-intimal pocket creation, prior to completing the subsequent stages of the valve securement. As shown in FIG. 17C, the pocket probe 32 may include an additional imaging element 608, which is distal to the balloon 38. This positioning of the imaging element 608 allows one to image actuation of the valve securement mechanism 48.

FIG. 17D depicts the sub-intimal pocket probe 32 being utilized. Following the first stage of actuation of the valve securement mechanism 48, and with the entire deflated pocket creation balloon 38 immersed within the inter-layer plane 31, the pocket creation balloon 38 is inflated through the inflation lumen 39, prompting expansion to its asymmetric shape. As depicted, the balloon expansion within the inter-layer plane 31 acts to further separate the inner layer tissue flap 63 from the outer layer 61 of the lumen wall, until a full sub-intimal pocket 64 has been created between the layers 61, 62. The geometry of this sub-intimal pocket 64 is determined by the shape, size and position of the pocket creation balloon 38 upon inflation. At this point, there exists a narrow inlet 65 in the top of the sub-intimal pocket with a circular shape just large enough to allow for dimensional clearance of the sub-intimal pocket probe 32. This inlet was created originally when the tissue engagement mechanism 23 penetrates through the vessel surface and into a wall of the vessel.

FIG. 17E depicts the second stage of actuation of the valve securement mechanism 48 immediately following, or simultaneously with, the sub-intimal pocket creation. After/during inflation of the pocket creation balloon 38, the securement mechanism delivery system 51 is further advanced, which acts to push the securement mechanism distal tip 53 through both the inner tissue layer 60 b and the outer tissue layer 61 b at the opposing side of the lumen, so that it rests in the extra-luminal space 66.

FIG. 17F depicts the third stage of actuation of the valve securement mechanism 48. Once the securement mechanism distal tip 53 has been advanced into the extra-luminal space 66, the constraining sheath 57 (not depicted) is retracted a small amount, allowing the distal clip arms 55 to spring outward into an orientation perpendicular to the axis of the delivery system 51 as a result of their shape memory characteristics. This clip orientation restricts the distal tip 53 from inadvertently disengaging in the backwards direction from the tissue layers through which it has been advanced.

FIG. 17G depicts the forth stage of actuation of the valve securement mechanism 48. The constraining sheath 57 (not depicted) is retracted further to allow the proximal clip arms 56 to spring outward into an orientation perpendicular to the axis of the delivery system 51 as a result of their shape memory characteristics. Once expanded, the proximal clip arms 56 rest within the sub-intimal pocket 64. At this point the inner tissue layer 60 a from one side of the lumen, the inner tissue layer 60 b from the other side of the lumen, and the outer tissue layer 61 b from the other side of the lumen, are constrained between the proximal clip arms 56 and the distal clip arms 55. Thus, the clip secures the flap formed from a first wall portion of a vessel relative to a second wall portion that is opposite from the first wall portion.

FIG. 17H depicts the fifth and final stage of actuation of the valve securement mechanism 48. The entire securement mechanism delivery system 51 is retracted forcing the securement mechanism distal tip 53 to detach from the securement mechanism delivery system 51 at the detachment joint 58. In this way, the securement mechanism distal tip 53 is left behind, depicted in this embodiment as an “H-tag” upon detachment. This form acts to prevent the newly separated inner tissue layer 60 a from assuming its natural orientation against the outer tissue layer 61 a, thus preventing it from biologically re-adhering in its original location. The delivery system 51 and the constraining sheath 57 are completely removed from the anatomy through the securement tool lumen 49 of the sub-intimal pocket probe 32. In other embodiments, instead of relying on tension to break the detachment joint 58, the joint 58 may be disintegratable in response to a current or heat applied therethrough.

FIG. 17I depicts a cross-section view of the bodily lumen 59 at the longitudinal position of the pocket creation balloon's 38 largest diameter (denoted A-A on FIG. 17H). A large percentage of the area of the bodily lumen is occupied by the newly created inter layer pocket 64.

FIG. 17J depicts a cross-section view of the bodily lumen 59 at the longitudinal position just proximal on the sub-intimal pocket probe 32 to the pocket creation balloon 38 (denoted B-B on FIG. 17H). At this location, the narrow inlet 65 at the top of the inner tissue layer flap 63 is seen, and is a much smaller opening than the full extent of the pocket diameter at a more distal location.

FIGS. 18A-18B depict the intimal separation mechanism 46 being utilized. The act of inflation of the pocket creation balloon 38 during the creation of a sub-intimal pocket 64 actuates the backward facing cutting mechanism 47 to its expanded orientation (depicted in FIG. 12F). As depicted in FIG. 18A, the sub-intimal pocket probe 46 is removed from the newly created sub-intimal pocket 64 while the pocket creation balloon 38 is still inflated. The imaging element 606 can be used to obtain images of the pocket surface as the pocket probe 46. The proximal movement of the mechanism 46 causes the cutting mechanism 47 to cut tissue next to the opening 65 at one end of the flap 63, thereby increasing the size of the opening 65. This provides the flap 63 with the desired width. It should be noted that as the inflated balloon 38 of the mechanism 46 is removed proximally out of the inlet 65, counter-tension is created at the vessel wall, which allows the backward facing cutting mechanism 47 to make a clean, consistent cut at the vessel wall along a path (e.g., a curved path) according to the shape of the expanded backward facing cutting mechanism 47. In the embodiment depicted, the circumferential angle (measured along the circumference of the vessel) of the inner tissue layer separation is over 180° (e.g., 180°+10°), and the cut is near horizontal (i.e., the direction of the cut is approximately perpendicular to the longitudinal axis of the vessel). In other embodiments, the cut may not be horizontal. Also, in other embodiments, the length of the cut made by the cutting mechanism 47 may be less than 180°. The separation of the inner tissue layer flap 63 finalizes the top lip of the newly created autologous valve 67. At this point, the pocket creation balloon 38 is deflated, and the autologous valve creation has been accomplished. FIG. 18B depicts a cross-sectional view of the fully created autologous valve 67 at the longitudinal plane located directly proximal to the securement of the inner tissue layer flap 63 (denoted B-B on FIG. 17H), after the device has been fully removed.

After the valve 67 is created, the user may visualize the effect of autologous valve creation using fluoroscopic visualization techniques. Contrast agent 10 can be injected through the forward facing exit port 25 of the pocket-creation mechanism 32 (or through another fluid delivery device) at any appropriate time during the procedure. This tool will be especially useful after valve creation has been accomplished. In this case, the user may first deflate the pocket creation balloon 38 to facilitate placement of the forward facing exit port 25 in the newly created sub-intimal pocket 64. Standard techniques—including manual pumping of the calf muscle—can be used to force blood flow through the autologous valve 67 for evaluation. Once visualization confirms that autologous valve 67 is functioning properly, the device is removed from the bodily lumen.

Systems and methods of the invention may include one or more expandable members (also called expansion elements). Typically, the expandable members are balloons. Balloons suitable for use in the invention may include any material that exhibits suitable strength and elasticity. Suitable materials may include polyvinyl chloride (PVC), cross-linked polyethylene (PET), nylon, or other polymers. In some embodiments, the balloon includes artificial muscle (electro-active polymer). Electro-active polymers exhibit an ability to change dimension in response to electric stimulation. The change may be driven by electric field E or by ions. Exemplary polymers that respond to electric fields include ferroelectric polymers (commonly known polyvinylidene fluoride and nylon 11, for example), dielectric EAPs, electro-restrictive polymers such as the electro-restrictive graft elastomers and electro-viscoelastic elastomers, and liquid crystal elastomer composite materials. Ion responsive polymers include ionic polymer gels, ionomeric polymer-metal composites, conductive polymers and carbon nanotube composites. Common polymer materials such as polyethylene, polystyrene, polypropylene, etc., can be made conductive by including conductive fillers to the polymer to create current-carrying paths. Many such polymers are thermoplastic, but thermosetting materials such as epoxies, may also be employed. Suitable conductive fillers include metals and carbon, e.g., in the form of sputter coatings. Electro-active polymers are discussed in U.S. Pat. No. 7,951,186; U.S. Pat. No. 7,777,399; and U.S. Pub. 2007/0247033, the contents of each of which are incorporated by reference. Balloons can be inflated using any technique known in the art, typically by introducing a fluid or gaseous element into the balloon.

According to certain embodiments, the components of the endoluminal valve catheter systems of the invention include one or more imaging elements. Imaging elements of any one component may different from any other component. For example, the imaging element of a support catheter may be different from the puncture member, access probe, or pocket probe. Imaging elements suitable for use with components of the endoluminal valve catheter systems of the invention are described hereinafter. Typically, the imaging element is a component of an imaging assembly. Any imaging assembly may be used with devices and methods of the invention, such as optical-acoustic imaging apparatus, intravascular ultrasound (IVUS) or optical coherence tomography (OCT). The imaging element may be a forward looking imaging element or a side-looking imaging element. The imaging element is used to send and receive signals to and from the imaging surface that form the imaging data. All of the imaging elements described hereinafter may be coupled to a signal line that provide power and allow data transmission to and from the imaging element. Typically, the signal line is coupled to an imaging system, such as a computer. The signal lines may be routed through lumens already existing in components of the endoluminal valve catheter system. Alternatively, the components can be specifically designed with lumens, in which the one or more signal lines are routed therethrough. The creation of multi-lumen catheter components is known in the art.

The imaging assembly may be an intravascular ultrasound (IVUS) imaging assembly. IVUS uses an ultrasound probe attached at the distal end. The ultrasound probe is typically an array of circumferentially positioned transducers. However, it is also envisioned that the imaging element may be a rotating transducer. For example, when the puncture element is coupled to a rotary drive shaft to enable rotation of the puncture element, the imaging element may be a rotating transducer. The proximal end of the catheter is attached to computerized ultrasound equipment. The IVUS imaging element (i.e. ultrasound probe) includes transducers that image the tissue with ultrasound energy (e.g., 20-50 MHz range) and image collectors that collect the returned energy (echo) to create an intravascular image. The imaging transducers and imaging collectors are coupled to signal lines that run through the length of the catheter and couple to the computerized ultrasound equipment.

IVUS imaging assemblies produce ultrasound energy and receive echoes from which real time ultrasound images of a thin section of the blood vessel are produced. The imaging transducers of the imaging element are constructed from piezoelectric components that produce sound energy at 20-50 MHz. The image collectors of the imaging element comprise separate piezoelectric elements that receive the ultrasound energy that is reflected from the vasculature. Alternative embodiments of imaging assembly may use the same piezoelectric components to produce and receive the ultrasonic energy, for example, by using pulsed ultrasound. That is, the imaging transducer and the imaging collectors are the same. Another alternative embodiment may incorporate ultrasound absorbing materials and ultrasound lenses to increase signal to noise.

IVUS data is typically gathered in segments where each segment represents an angular portion of an IVUS image. Thus, it takes a plurality of segments (or a set of IVUS data) to image an entire cross-section of a vascular object. Furthermore, multiple sets of IVUS data are typically gathered from multiple locations within a vascular object (e.g., by moving the transducer linearly through the vessel). These multiple sets of data can then be used to create a plurality of two-dimensional (2D) images or one three-dimensional (3D) image.

IVUS imaging assemblies and processing of IVUS data are described in further detail in, for example, Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, U.S. Pub. 2009/0284332; U.S. Pub. 2009/0195514 A1; U.S. Pub. 2007/0232933; and U.S. Pub. 2005/0249391 and other references well known in the art relating to intraluminal ultrasound devices and modalities.

In other embodiments, the imaging assembly may be an optical coherence tomography imaging assembly. OCT is a medical imaging methodology using a miniaturized near infrared light-emitting probe. As an optical signal acquisition and processing method, it captures micrometer-resolution, three-dimensional images from within optical scattering media (e.g., biological tissue). Recently it has also begun to be used in interventional cardiology to help diagnose coronary artery disease. OCT allows the application of interferometric technology to see from inside, for example, blood vessels, visualizing the endothelium (inner wall) of blood vessels in living individuals.

OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

In OCT, a light source delivers a beam of light to an imaging device to image target tissue. Light sources can include pulsating light sources or lasers, continuous wave light sources or lasers, tunable lasers, broadband light source, or multiple tunable laser. Within the light source is an optical amplifier and a tunable filter that allows a user to select a wavelength of light to be amplified. Wavelengths commonly used in medical applications include near-infrared light, for example between about 800 nm and about 1700 nm.

Aspects of the invention may obtain imaging data from an OCT system, including OCT systems that operate in either the time domain or frequency (high definition) domain. Basic differences between time-domain OCT and frequency-domain OCT is that in time-domain OCT, the scanning mechanism is a movable mirror, which is scanned as a function of time during the image acquisition. However, in the frequency-domain OCT, there are no moving parts and the image is scanned as a function of frequency or wavelength.

In time-domain OCT systems an interference spectrum is obtained by moving the scanning mechanism, such as a reference mirror, longitudinally to change the reference path and match multiple optical paths due to reflections within the sample. The signal giving the reflectivity is sampled over time, and light traveling at a specific distance creates interference in the detector. Moving the scanning mechanism laterally (or rotationally) across the sample produces two-dimensional and three-dimensional images.

In frequency domain OCT, a light source capable of emitting a range of optical frequencies excites an interferometer, the interferometer combines the light returned from a sample with a reference beam of light from the same source, and the intensity of the combined light is recorded as a function of optical frequency to form an interference spectrum. A Fourier transform of the interference spectrum provides the reflectance distribution along the depth within the sample.

Several methods of frequency domain OCT are described in the literature. In spectral-domain OCT (SD-OCT), also sometimes called “Spectral Radar” (Optics letters, Vol. 21, No. 14 (1996) 1087-1089), a grating or prism or other means is used to disperse the output of the interferometer into its optical frequency components. The intensities of these separated components are measured using an array of optical detectors, each detector receiving an optical frequency or a fractional range of optical frequencies. The set of measurements from these optical detectors forms an interference spectrum (Smith, L. M. and C. C. Dobson, Applied Optics 28: 3339-3342), wherein the distance to a scatterer is determined by the wavelength dependent fringe spacing within the power spectrum. SD-OCT has enabled the determination of distance and scattering intensity of multiple scatters lying along the illumination axis by analyzing a single the exposure of an array of optical detectors so that no scanning in depth is necessary. Typically the light source emits a broad range of optical frequencies simultaneously.

Alternatively, in swept-source OCT, the interference spectrum is recorded by using a source with adjustable optical frequency, with the optical frequency of the source swept through a range of optical frequencies, and recording the interfered light intensity as a function of time during the sweep. An example of swept-source OCT is described in U.S. Pat. No. 5,321,501.

Generally, time domain systems and frequency domain systems can further vary in type based upon the optical layout of the systems: common beam path systems and differential beam path systems. A common beam path system sends all produced light through a single optical fiber to generate a reference signal and a sample signal whereas a differential beam path system splits the produced light such that a portion of the light is directed to the sample and the other portion is directed to a reference surface. Common beam path systems are described in U.S. Pat. No. 7,999,938; U.S. Pat. No. 7,995,210; and U.S. Pat. No. 7,787,127 and differential beam path systems are described in U.S. Pat. No. 7,783,337; U.S. Pat. No. 6,134,003; and U.S. Pat. No. 6,421,164, the contents of each of which are incorporated by reference herein in its entirety.

In yet another embodiment, the imaging assembly is an optical-acoustic imaging apparatus. Optical-acoustic imaging apparatus include at least one imaging element to send and receive imaging signals. In one embodiment, the imaging element includes at least one acoustic-to-optical transducer. In certain embodiments, the acoustic-to-optical transducer is an Fiber Bragg Grating within an optical fiber. In addition, the imaging elements may include the optical fiber with one or more Fiber Bragg Gratings (acoustic-to-optical transducer) and one or more other transducers. The at least one other transducer may be used to generate the acoustic energy for imaging. Acoustic generating transducers can be electric-to-acoustic transducers or optical-to-acoustic transducers. The imaging elements suitable for use in devices of the invention are described in more detail below.

Fiber Bragg Gratings for imaging provides a means for measuring the interference between two paths taken by an optical beam. A partially-reflecting Fiber Bragg Grating is used to split the incident beam of light into two parts, in which one part of the beam travels along a path that is kept constant (constant path) and another part travels a path for detecting a change (change path). The paths are then combined to detect any interferences in the beam. If the paths are identical, then the two paths combine to form the original beam. If the paths are different, then the two parts will add or subtract from each other and form an interference. The Fiber Bragg Grating elements are thus able to sense a change wavelength between the constant path and the change path based on received ultrasound or acoustic energy. The detected optical signal interferences can be used to generate an image using any conventional means.

Exemplary optical-acoustic imaging assemblies are disclosed in more detail in U.S. Pat. Nos. 6,659,957 and 7,527,594, 7,245.789, 7447,388, 7,660,492, 8,059,923 and in U.S. Patent Publication Nos. 2008/0119739, 2010/0087732 and 2012/0108943.

In certain embodiments, an imaging element is disposed beneath or on a surface of an expansion member or balloon.

The imaging element may be a side-looking imaging element, a forward-looking imaging element, or combination thereof. Examples of forward-looking ultrasound assemblies are described in U.S. Pat. Nos. 7,736,317, 6,780,157, and 6,457,365, and in Yao Wang, Douglas N. Stephens, and Matthew O'Donnellie, “Optimizing the Beam Pattern of a Forward-Viewing Ring-Annular Ultrasoun Array for Intravascular Imaging”, Transactions on Ultrasonics, Rerroelectrics, and Frequency Control, vol. 49, no. 12, December 2002. Examples of forward-looking optical coherence tomography assemblies are described in U.S. Publication No. 2010/0220334, Fleming C. P., Wang H., Quan, K. J., and Rollins A. M., “Real-time monitoring of cardiac radio-frequency ablation lesion formation using an optical coherence tomography forward-imaging catheter.,” J. Biomed. Opt. 15, (3), 030516-030513 ((2010)), and Wang H, Kang W, Carrigan T, et al; In vivo intracardiac optical coherence tomography imaging through percutaneous access: toward image-guided radio-frequency ablation. J. Biomed. Opt. 0001; 16(11):110505-110505-3. doi:10.1117/1.3656966. In certain aspects, an imaging assembly includes both side-viewing and forward-looking capabilities. These imaging assemblies utilize different frequencies that permit the imaging assembly to isolate between forward looking imaging signals and side viewing imaging signals. For example, the imaging assembly is designed so that a side imaging port is mainly sensitive to side-viewing frequencies and a forward viewing imaging port is mainly sensitive to forward viewing frequencies. Example of this type of imaging element is described in U.S. Pat. Nos. 7,736,317, 6,780,157, and 6,457,365.

Functional measurement sensors suitable coupled to one or more components of endoluminal valve catheter systems of the invention include, for example, a pressure sensor, temperature sensors, flow sensor, or combination thereof.

A pressure sensor allows one to obtain pressure measurements within a body lumen. A particular benefit of pressure sensors is that pressure sensors allow one to measure of FFR in vessel. FFR is a comparison of the pressure within a vessel at positions prior to the stenosis and after the stenosis. The level of FFR determines the significance of the stenosis, which allows physicians to more accurately identify clinically relevant stenosis. For example, an FFR measurement above 0.80 indicates normal coronary blood flow and a non-significant stenosis. Another benefit is that a physician can measure the pressure before and after an intraluminal intervention procedure to determine the impact of the procedure.

A pressure sensor can be mounted on the distal portion of a flexible elongate member. In certain embodiments, the pressure sensor is positioned distal to the compressible and bendable coil segment of the elongate member. This allows the pressure sensor to move along with the along coil segment as bended and away from the longitudinal axis. The pressure sensor can be formed of a crystal semiconductor material having a recess therein and forming a diaphragm bordered by a rim. A reinforcing member is bonded to the crystal and reinforces the rim of the crystal and has a cavity therein underlying the diaphragm and exposed to the diaphragm. A resistor having opposite ends is carried by the crystal and has a portion thereof overlying a portion of the diaphragm. Electrical conductor wires can be connected to opposite ends of the resistor and extend within the flexible elongate member to the proximal portion of the flexible elongate member. Additional details of suitable pressure sensors that may be used with devices of the invention are described in U.S. Pat. No. 6,106,476. U.S. Pat. No. 6,106,476 also describes suitable methods for mounting the pressure sensor 104 within a sensor housing.

A flow sensor can be used to measure blood flow velocity within the vessel, which can be used to assess coronary flow reserve (CFR). The flow sensor can be, for example, an ultrasound transducer, a Doppler flow sensor or any other suitable flow sensor, disposed at or in close proximity to the distal tip of the guidewire. The ultrasound transducer may be any suitable transducer, and may be mounted in the distal end using any conventional method, including the manner described in U.S. Pat. Nos. 5,125,137, 6,551,250 and 5,873,835.

External imaging modality devices for use in methods and devices of the invention include, for example, X-ray angiography imaging, computed tomography imaging, and magnetic resonance imaging devices. Preferably, the imaging modality is computed tomography which does not require the use of a contrast, which may not enter the small vessels of the microvasculature or stenosis vessels in adequate amounts for proper imaging.

In some embodiments, a device of the invention includes an imaging assembly and obtains a three-dimensional data set through the operation of OCT, IVUS, or other imaging hardware. In addition, a device of the invention, according to certain embodiments, may include a functional measurement sensor that obtains data through operation of functional measurement hardware. The imaging hardware and functional measurement hardware may be the same or different. In some embodiments, a device of the invention is a computer device such as a laptop, desktop, or tablet computer, and obtains a three-dimensional data set by retrieving it from a tangible storage medium, such as a disk drive on a server using a network or as an email attachment.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

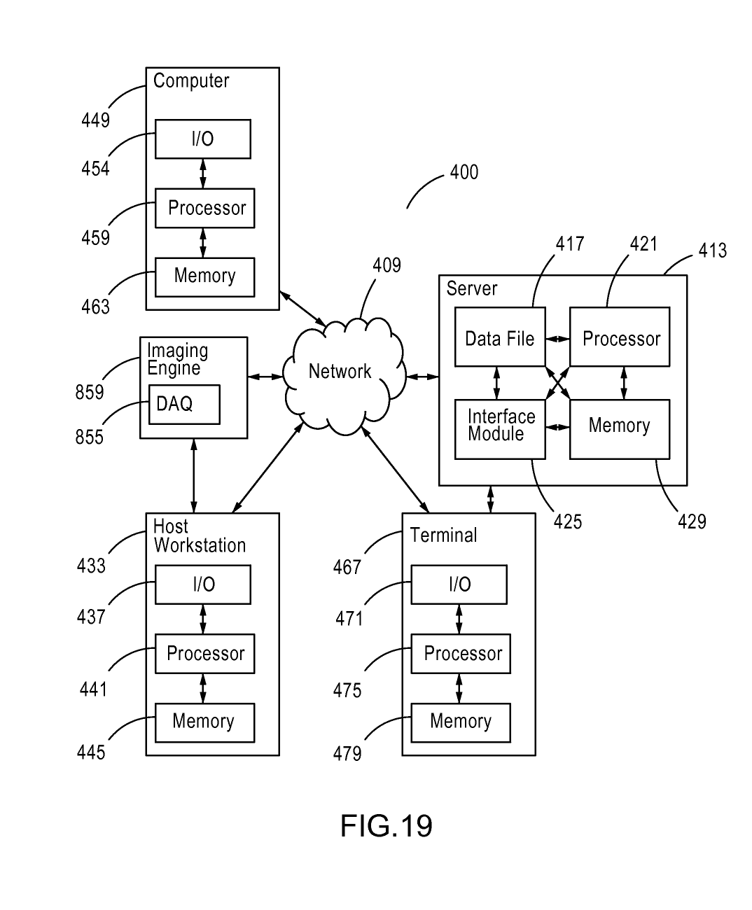

In some embodiments, a user interacts with a visual interface to view images from the imaging system. Input from a user (e.g., parameters or a selection) are received by a processor in an electronic device. The selection can be rendered into a visible display. An exemplary system including an electronic device is illustrated in FIG. 19. As shown in FIG. 19, an imaging engine 859 of the imaging assembly communicates with host workstation 433 as well as optionally server 413 over network 409. The data acquisition element 855 (DAQ) of the imaging engine receives imaging data from one or more imaging element. In some embodiments, an operator uses computer 449 or terminal 467 to control system 400 or to receive images. An image may be displayed using an I/O 454, 437, or 471, which may include a monitor. Any I/O may include a keyboard, mouse or touchscreen to communicate with any of processor 421, 459, 441, or 475, for example, to cause data to be stored in any tangible, nontransitory memory 463, 445, 479, or 429. Server 413 generally includes an interface module 425 to effectuate communication over network 409 or write data to data file 417.

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server 413), a middleware component (e.g., an application server), or a front-end component (e.g., a client computer 449 having a graphical user interface 454 or a web browser through which a user can interact with an implementation of the subject matter described herein), or any combination of such back-end, middleware, and front-end components. The components of the system can be interconnected through network 409 by any form or medium of digital data communication, e.g., a communication network. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a portion of file 417 that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over network 409 (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

In addition, system and methods of the invention provide an implantable valve. The implantable valve is an artificial valve prosthesis designed to replace or supplement the function of incompetent valve. The valve prostheses of the invention are constructed so as to allow fluid flow in a first, antegrade, direction and to restrict fluid flow in a second, retrograde, direction.

Implantable valves of the invention are desirably adapted for deployment within a body lumen, and in particular embodiments, devices and systems of the invention are adapted for deployment within the venous system. Accordingly, preferred devices adapted are venous valves, for example, for percutaneous implantation within veins of the legs or feet to treat venous insufficiency. However, devices and systems of the present invention may be adapted for deployment within any tube-shaped body passage lumen that conducts fluid, including but not limited to blood vessels, such as those of the human vasculature system; billiary ducts; ureteral passages and the alimentary canal.

One aspect of the present invention provides a self-expanding or otherwise expandable artificial valve prosthesis for deployment within a bodily passageway, such as a vessel or duct of a patient. The prosthesis is typically delivered and implanted using well-known transcatheter techniques for self-expanding or otherwise expandable prostheses. The valve prosthesis is positioned so as to allow antegrade fluid flow and to restrict retrograde fluid flow. Antegrade fluid flow travels from the distal (upstream) end of the prosthesis to the proximal (downstream) end of the prosthesis, the latter being located closest to the heart in a venous valve when placed within the lower extremities of a patient. Retrograde fluid flow travels from the proximal (downstream) end of the prosthesis to the distal (upstream) end of the prosthesis

The implantable valves of the invention may be delivered into a body lumen using a delivery catheter. Delivery catheters are known in the art. Exemplary delivery catheters include those described in U.S. Pat. Nos. 8,167,932 8,021,420, 8,475,522 and 8,353,945 as well as U.S. Publication Nos. 2012/0310332 and 2012/029007.

Prior art valves generally include one or more leaflets that allow blood flow traveling towards the heart, but close to prevent blood flow traveling away from the heart. A problem with prior art implantable valves is that the valves create unnatural pressure build up during the complete restriction of blood flow traveling away from the heart. Unlike prior art prosthetic valves, natural valves are able to avoid pressure build up in the veins while still restricting undesirable volumes of fluid flow away from the heart.

The present invention solves this problem by providing a valve with two or more leaflets supported by a frame that form a central opening. The valve is deformable between a first position allowing fluid flow in a first direction through the central opening, and a second position restricting fluid flow in the second direction. While in the second position, the leaflets close the central opening. At least one of the leaflets includes a plurality of openings on the body of the leaflet. The plurality of openings allows minor fluid flow in the first and second direction in order to prevent undesirable pressure build up. Thus, valves of the invention allow fluid flow through the central opening of a first volume, and fluid flow through the plurality of openings of a second volume. The first volume is greater than the second volume. The amount of openings and the size of openings formed in a body of one or more valve leaflets can be chosen depending on the desired amount of fluid flow in both directions when the valve is in the restricted position.

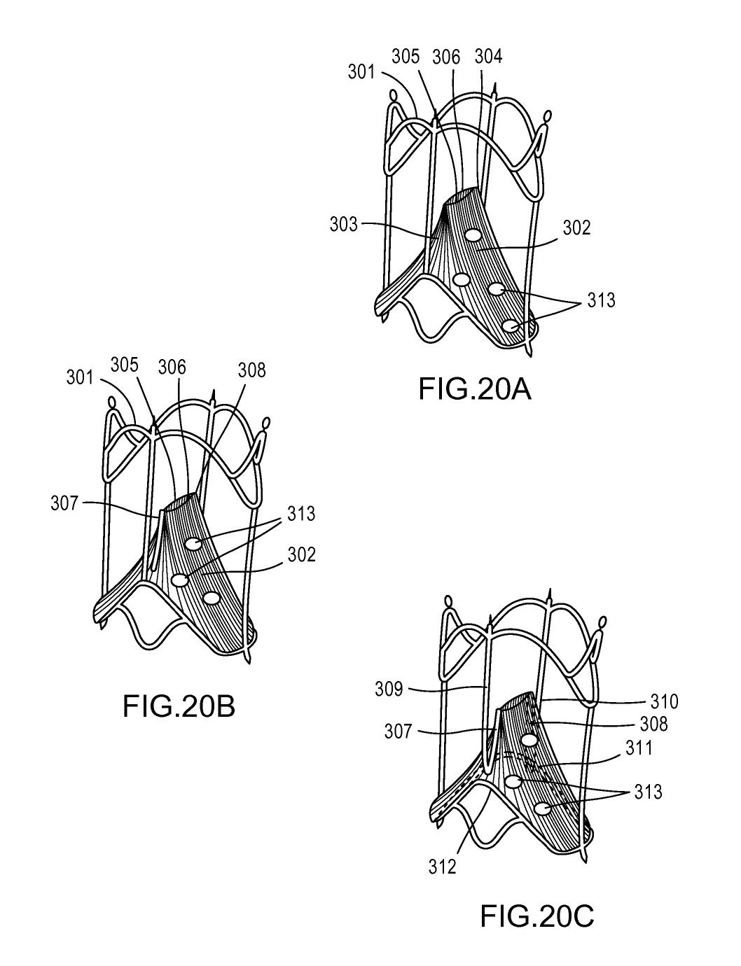

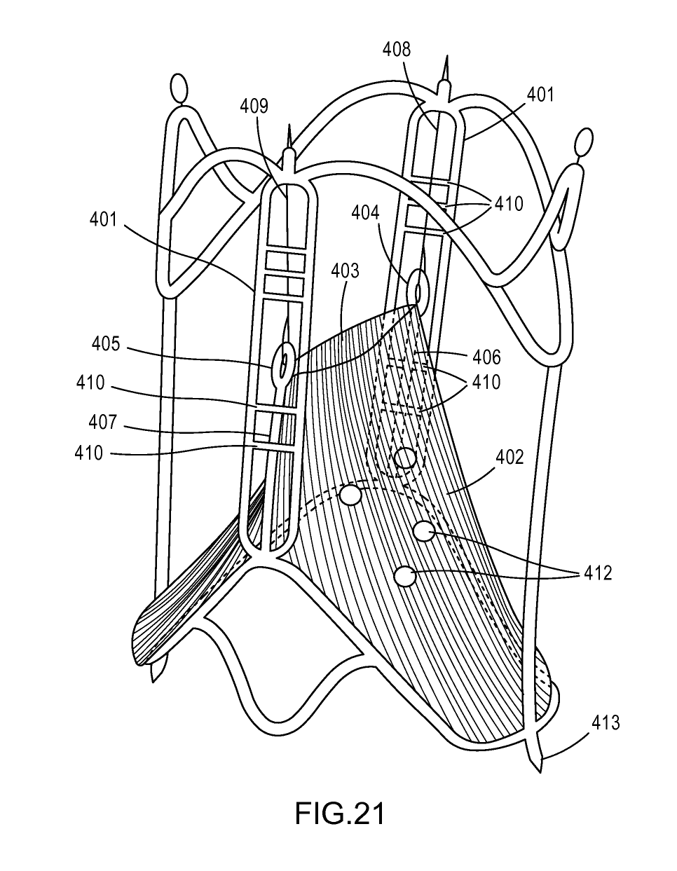

FIG. 20A shows an illustrative embodiment of an implantable valve of the present invention. Support frame 301 supports two valve leaflets formed from a continuous membrane 302 in the form of a cone structure that attaches to support frame 301 towards the upstream end of the valve prosthesis. The cone structure tapers towards the downstream end of the valve prosthesis and terminates at two co-apting edges 305 and 306. The length of these edges is shorter that the expanded diameter of the support frame. In one embodiment, reinforced portions 303 and 304 may be incorporated into the cone structure to help support the cone structure and prevent prolapse.

FIG. 20B shows yet another illustrative embodiment of the valve prosthesis of the present invention. Here, the cone structure 302 is supported by support elements 307 and 308. In this embodiment, support elements 307 and 308 do not attach to support structure 301. Again, the length of co-apting edges 305 and 306 is shorter that the expanded diameter of the support frame. Support elements 307 and 308 accommodate limited radial movement of commissural point with respect to support frame 301. For example, support elements 307 and 308 can be flexible itself or can be attached to support frame 301 by a flexible join. For example, the join may include a coil or a fillet, although a simple bend may offer superior fatigue life for some materials. Example of frames having such joins may be found in U.S. Publication No. 2004/0186558, published Sep. 23, 2004, the contents of which are incorporated by reference. Such a configuration allows support element 307, 308 to accommodate sealing along the co-apting edges of valve leaflets 302, when subjected to retrograde flow, even when the fully expanded radial diameter of support frame 301 is slightly oversized relative to the body vessel in which the valve prosthesis is placed.

FIG. 20C shows another illustrative embodiment. In this embodiment, support elements 307 and 308 attach to support frame members 309 and 310 respectively but not to support frame members 311 and 312.

As shown in FIG. 20A-20C, at least one of the leaflets of the valve 302 includes a plurality of openings 313 on the body of the leaflet that allow some fluid flow in the antegrade and retrograde direction.

FIG. 21 shows an illustrative embodiment of the implantable valve in which support elements 406 and 407 are additionally supported by elements 408 and 409 respectively. For example, elements 408 and/or 409 may be sutures. Support elements 406 and 407 may contain eyelets 404 and 405 to provide anchoring points for the sutures. Alternatively, elements 408 and 409 may be similar to support elements 406 and 407. In one embodiment, support frame 401 may include rib elements 410. Such elements limit or prevent the collapse of the vessel wall through the support frame onto support elements 406 and 407 and valve leaflets 402 and 403. The at least one valve leaflets 402 and 403 includes a plurality of openings 312 on the body of the leaflet 402, 403 that allow some fluid flow in the antegrade and retrograde direction.

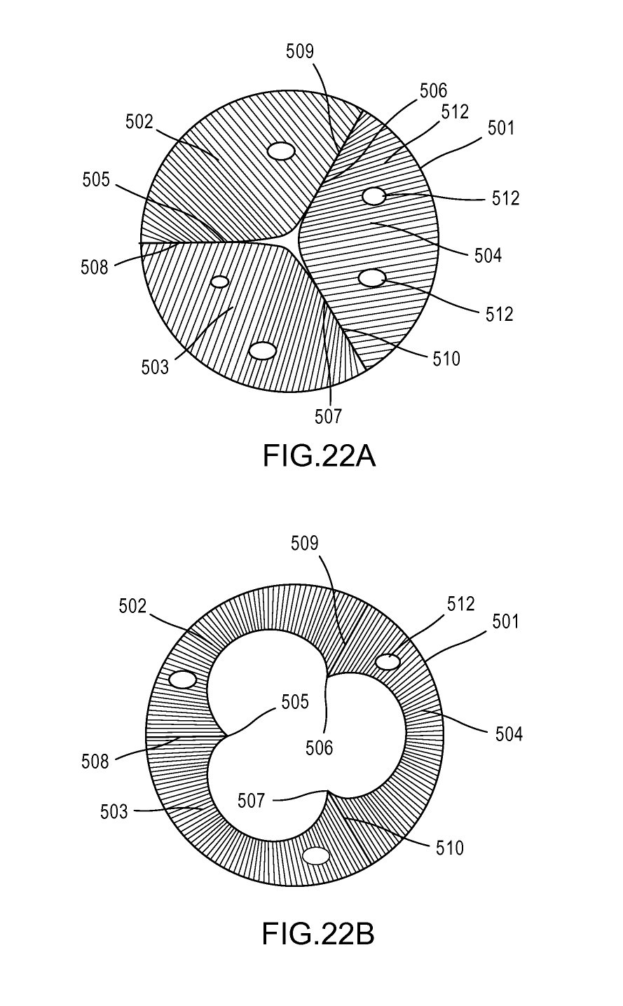

FIGS. 22(a) and 22(b) shows a plan view of another illustrative embodiment of an implantable valve. Support frame 501 and support elements 508, 509 and 510 support three valve leaflets 502, 503 and 504 that co-apt between three commissural points 505, 506 and 507. The regions of the perimeter of the valve leaflets supported by support elements 508, 509 and 510 are again positioned away from the vessel wall. FIG. 22(a) shows the valve leaflets restricting retrograde flow while FIG. 22(b) shows the valve leaflets positioned in an open position by antegrade flow. As shown in FIGS. 22A-22B, the valve leaflets include a plurality of openings that allow some fluid flow in the antegrade and retrograde direction.

In the above embodiments, the amount of slack in the valve leaflet material determines, at least in part, how well the valve leaflets restrict retrograde flow and how large of an opening they permit during antegrade flow. In one embodiment, the valve prosthesis is configured such that, when the valve leaflets are positioned in their fully open position by antegrade flow, the cross sectional area available for fluid flow is between 90 and 10 percent of the cross sectional area of the expanded outer frame in the region of attachment of the valve leaflets to the support frame. In another embodiment, the valve prosthesis is configured such that the cross sectional area available for antegrade fluid flow is between 70 and 30 percent of the cross sectional area of the expanded outer frame in the region of attachment of the valve leaflets to the support frame. In yet another embodiment, the valve prosthesis is configured such that the cross sectional area available for antegrade fluid flow is between 50 and 40 percent of the cross sectional area of the expanded outer frame in the region of attachment of the valve leaflets to the support frame.

Elements shown in the embodiments described herein can be added to and/or exchanged with other embodiments to provide additional embodiments. It will also be understood that other valve body configurations are also contemplated as being within the scope of the present invention. For example, valves having four or more valve leaflets are contemplated. Hence, the number of leaflets possible for embodiments of the present invention can be one, two, three, four, five or any practical number. Bi-leaflet valves are preferred in low-flow venous situations. The valve leaflets may be of equal size and shape or of differing size and shape depending on the configuration of the supporting frame members.

The support frame used in the artificial valve prosthesis of the present invention can be, for example, formed from wire, cut from a section of cannula, molded or fabricated from a polymer, biomaterial, or composite material, or a combination thereof. The pattern (i.e., configuration of struts and cells) of the outer frame, including any anchoring portion(s), which is selected to provide radial expandability to the prosthesis is also not critical for an understanding of the invention. Any support frame is applicable for use with the claimed valve prosthesis so long as this structure allows the valve leaflets to be supported in the required position and allows the required portion of the perimeter of the leaflet to remain away from the vessel wall. Numerous examples of support structures are disclosed in U.S. Patent Publication No. 2004/01866558A1, published Sep. 23, 2004, the contents of which are incorporated herein by reference. In certain embodiments, the support frame includes one or more hooks to stabilize the support frame within the vessel, such as the hook 413 depicted in FIG. 21.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A system for endoluminal valve formation, the system comprising
an elongate body defining a lumen and an exit port located on a side of the elongate body, wherein the side of the elongate body is configured to engage with a vessel wall;
a tissue dissection probe disposed within the lumen, and configured to extend out of the exit port and into the vessel wall in order to form an intramural space in the vessel wall; and
an imaging element located on the tissue dissection probe.

2. The system of claim 1, wherein the elongate body comprises a distal portion, and the tissue dissection probe is configured to distally extend out of the exit port in an orientation that is substantially parallel with the distal portion.

3. The system of claim 2, wherein a cross-section of the distal portion is smaller than a cross-section of the elongate body proximal to the distal portion.

4. The system of claim 1, wherein the tissue dissection probe defines a lumen terminating at a distal opening and is operably associated with a mechanism configured to deliver hydro-dissection fluid from the opening.

5. The system of claim 1, wherein the imaging element is selected from the group consisting of a photoacoustic transducer and an ultrasound transducer.

6. The system of claim 5, wherein the ultrasound transducer is an array-based transducer.

7. The system of claim 5, wherein the ultrasound transducer is a forward-looking transducer.

8. The system of claim 1, wherein the tissue dissection probe comprises an expandable member.

9. The system of claim 8, wherein the imaging element is disposed on the expandable member.

10. The system of claim 8, wherein the imaging element is disposed within the expandable member.

11. A catheter for endoluminal valve formation, the catheter comprising
a catheter body defining a lumen and configured to enter a vessel;
a distal portion of the catheter body configured to engage with a wall of the vessel;
an exit port along a side of the catheter body and proximal to the distal portion; and
a tissue dissection probe disposed within the catheter lumen and comprising an imaging element, wherein the tissue dissection probe is configured to distally extend out of the exit port and into a wall of the vessel.

12. The catheter of claim 11, wherein the tissue dissection probe is configured to distally extend out of the exit port in an orientation that is substantially parallel with the distal portion.

13. The catheter of claim 11, wherein a cross-section of the distal portion is smaller than a cross-section of the catheter body proximal to the distal portion.

14. The catheter of claim 11, wherein the tissue dissection probe defines a lumen terminating at a distal opening and is operably associated with a mechanism configured to deliver hydro-dissection fluid from the opening.

15. The catheter of claim 11, wherein the imaging element is selected from the group consisting of a photoacoustic transducer and an ultrasound transducer.

16. The catheter of claim 15, wherein the ultrasound transducer is an array-based transducer.

17. The catheter of claim 15, wherein the ultrasound transducer is a forward-looking transducer.

18. The catheter of claim 11, wherein the tissue dissection probe comprises an expandable member.

19. The catheter of claim 18, wherein the imaging element is disposed on the expandable member.

20. The catheter of claim 18, wherein the imaging element is disposed within the expandable member.

21. The catheter of claim 18, wherein the expandable member comprises a first end and a second end, and, when expanded, the first end defines a volume greater than the second end.

22. The catheter of claim 18, wherein the expandable member, when expanded, defines a conical volume.

23. A method for forming an endoluminal valve, the method comprising
introducing a catheter into a lumen of a vessel;
advancing the catheter to a location for endoluminal valve formation within the vessel;
extending a tissue dissection probe from the catheter and into a wall of the vessel at the location; wherein the tissue dissection probe comprises a imaging element;
forming, with the tissue dissection probe, an intramural space within the wall of the vessel, thereby forming a tissue flap; and
imaging, with the tissue dissection probe, the forming step.

24. The method of claim 23, wherein the forming step comprises
delivering hydro-dissection fluid from a distal end of the tissue dissection probe into the vessel wall.

25. The method of claim 23, wherein the forming step further comprises
expanding an expandable member located on a portion of the tissue dissection probe disposed within the vessel wall.

26. The method of claim 23, wherein the imaging element is selected from the group consisting of a photoacoustic transducer and an ultrasound transducer.
